(19) United States
(12) Patent Application Publication
(10) Pub. No.: US 2015/0099709 A1
(43) Pub. Date: Apr. 9, 2015

(54) GHRELIN RECEPTOR AGONISTS FOR THE TREATMENT OF ACHLORHYDRIA

(71) Applicant: RaQualia Pharma Inc., Aichi (JP)

(72) Inventors: Nobuyuki Takahashi, Aichi (JP); Masaki Sudo, Aichi (JP); Kaoru Shimada, Aichi (JP); Hiroaki Wakabayashi, Aichi (JP)

(21) Appl. No.: 14/400,194

(22) PCT Filed: May 27, 2013
(86) PCT No.: PCT/JP2013/003331

§ 371 Date: Nov. 10, 2014

Related U.S. Application Data
(60) Provisional application No. 61/651,662, filed on May 25, 2012.

(51) Int. Cl. A61K 38/12; A61K 31/45; A61K 31/444; A61K 31/454; A61K 31/437; A61K 45/06

(52) U.S. Cl. CPC A61K 38/12; A61K 31/437; A61K 45/06; A61K 31/444; A61K 31/454; A61K 31/45
USPC 514/21.9; 514/303; 546/120; 514/323; 546/201; 514/315; 546/221; 530/331

(57) ABSTRACT

The present invention relates to a use of a compound having ghrelin receptor agonistic activity, a pharmaceutically acceptable salt thereof or a pharmaceutical composition thereof for the manufacture of a medicament for treatment of diseases including achlorhydria in which abnormal gastric acid secretion is involved. In addition, the present invention relates to the method of treatment including administering to a human or animal. The compound, the pharmaceutically acceptable salt thereof, or pharmaceutical compositions containing them, may be used in combination with one or more second active agents. Further, the present invention relates to pharmaceutical compositions and kits comprising a compound of the present invention or a pharmaceutically acceptable salt thereof for the treatment of said diseases.

### TECHNICAL FIELD

The present invention relates to providing a drug to increase gastric-acid secretion. Specifically, this invention relates to use of a compound which has agonistic activities against ghrelin receptor or a pharmaceutically acceptable salt thereof, or a pharmaceutical composition comprising the compound or the salt for the manufacture of a medicament for the treatment of diseases including achlorhydria in which abnormal gastric acid secretion is involved. The invention relates to use of the said compound, or a pharmaceutically acceptable salt thereof, or a pharmaceutical composition comprising the compound or the salt, optionally in combination with one or more second active agents.

The invention relates to a method for the treatment of diseases including achlorhydria, which abnormal gastric acid secretion is involved in, comprising administering the compound of the present invention or a pharmaceutical composition comprising the same to humans or animals.

Further, this invention relates to a pharmaceutical composition or a kit comprising the compound of the present invention or a pharmaceutically acceptable salt thereof for the treatment of said diseases.

### BACKGROUND ART

Digestive tract disorders are one of the common diseases in routine clinical practice. For the treatment of diseases in which high gastric acid secretion is involved, gastric acid secretion inhibitors (dried aluminum hydroxide gel, magnesium oxide, etc.), antipeptic drugs (sucralfate, ecabet sodium, etc.), gastric acid secretion inhibitors (anticholinergic drugs, H2 blockers, proton pump inhibitors, etc.) and the like are used clinically. Development of H2 blockers and proton pump inhibitors achieved ground-breaking progress in the treatment of upper digestive tract disorders.

On the other hand, for the treatment of diseases such as achlorhydria in which low or no gastric acid secretion is involved, no preferable drugs have been provided yet. Therefore as an agent for promoting the secretion of gastric acid or gastric fluid, 1) aromatic bitters, such as Swertia japonica, Picrasma quassioides, and Phellodendron amurense; 2) aromatics, such as Foeniculum vulgare, Cinnamomum cassia, 3) digestive fluids orenzymes, such as diastase, pepsin, amylase, and lipase; 4) acetylcholine derivatives; and 5) acids such as hydrochloric acid, citric acid, and tartaric acid have been used.

It has been known that ratio of people of achlorhydria which have intragastric pH>5.5 increases with aging and those in their 50s reached more than 60% (Journal of Pharmacobiodynamics, vol 7, 656-664, 1984).

As mentioned above, for gastric ulcers, duodenal ulcers, and other peptic ulcers, drugs (such as gastric-acid secretion blockers, and gastric antacids) that suppress digestive fluids and other visceral-wall invasive factors, and drugs (such as muco-protective agents) that reinforce defense mechanisms have been used. Nevertheless, although promoting gastric-acid secretion presumably should be effective in patients with chronic gastritis, particularly atrophic gastritis, under the existing circumstances, gastric promotorting drugs that are pharmacologically applicable to humans or animals have not been developed.

In addition, achlorhydria is observed in patients with anemic condition. The recognition that iron deficiency anemia was a long-term consequence of partial gastrectomy indicated the importance of gastric acid in the absorption of dietary iron. (Suzana Kovaca, Gregory J. Andersonb, Graham S. Baldwin, Biochimica et Biophysica Acta, Molecular Cell Research, Volume 1813, Issue 5, 889-895, May 2011).

It has been also pointed out that gastric acid secretion plays an important role in calcium absorption since both the dissociation of food-calcium complexes and the solution of calcium salts are highly dependent on an acid pH (Bo-Linn G W, Davis G R, Buddrus D J, Morawski S G, Santa Ana C and Fordran J S, J. Clin. Invest., 73:640-647, 1984; Nordin, B. E. C., Gastroenterology. 54:294-301, 1968; Ivanovich, P., H. Fellows, and C. Rich, The absorption of calcium carbonate. Ann. Intern. Med. 66:917-923, 1967.).

Performance of a gastrectomy may lead to other physiologic changes outside of reduced gastric acid production which may affect calcium absorption, including impaired vitamin D absorption, and defective calcitonin synthesis (Gertner J. M., Lilburn M., Domenec M., Br. Med. J., 1, 1310-1312, 1977; Filipponi P., Gregorio F., Cristallini S. et al. Partial gastrectomy and mineral metabolism: effects on gastrin-calcitonin release, Bone Miner., 11, 199-208, 1990).

Further, proton pump inhibitors (PPIs) are used primarily to treat gastroesophageal reflux disease. Proton pump inhibitor-induced achlorhydria increases circulating gastrin and chromogranin A (CGA). Chromogranin is a widely used biomarker for the diagnosis and follow-up for gut-based neuroendocrin (Raines D., Chester M., Diebold A. E., Mamikunian P., Anthony C. T., Mamikunian G., Woltering E. A., Pancreas. Pancreas., May; 41(4):508-11, 2012).

Many other drugs, in addition to PPIs, have been reported achlorhydria as a side effect. Examples of such drugs include amoxicillin, atorvastatin calcium, calcitriol, carboplatin, clofazimine, cyclosporine, digoxin, esomeprazole magnesium, famotidine, fluconazole, losartan potassium, methotrexate sodium, omeprazole, omeprazole magnesium, pamidronate disodium, pantoprazole sodium, quetiapine fumarate, quinidine gluconate, ranitidine, ranitidine hydrochloride, troglitazone, trovafloxacin mesylate, and zoledronic acid.

According to the line, great efforts have been made to find or prepare a compound for achlorhydria derived by various causes. However, no preferable drugs for achlorhydria have been provided yet.

### CITATION LIST

### Non Patent Literature

NPL 1: Journal of Pharmacobiodynamics, vol 7, 656-664, 1984.
NPL 2: Suzana Kovaca, Gregory J. Andersonb, Graham S. Baldwin, Biochimica et Biophysica Acta, Molecular Cell Research, Volume 1813, Issue 5, 889-895, May 2011.
NPL 3: Bo-Linn G. W., Davis G. R., Buddrus D. J., Morawski S G, Santa Ana C and Fordran J S, J. Clin. Invest., 73:640-647, 1984.
NPL 4: Nordin B. E. C., Gastroenterology, 54:294-301, 1968.
NPL 5: Ivanovich P., Fellows H., and Rich C., The absorption of calcium carbonate. Ann. Intern. Med., 66:917-923, 1967.
NPL 6: Gertner J. M., M. Lilburn, M. Domenec, Br. Med. J., 1, 1310-1312, 1977.
NPL 7: Filipponi P., Gregorio F., Cristallini S. et al., Bone Miner., 11, 199-208, 1990.
NPL 8: Raines D., Chester M., Diebold A. E., Mamikunian P., Anthony C. T., Mamikunian G., Woltering E. A., Pancreas. Pancreas., May; 41(4):508-11, 2012.

### SUMMARY OF INVENTION

### Technical Problem

Under the circumstances mentioned in the background art, there is a standing need for a compound or a composition that should ameliorate or inhibit progression of achlorhydric symptoms.

### Solution to Problem

Inventors of the present invention studied a group of compounds which were effective for increasing gastric acid secretion, and reached that a ghrelin receptor agonist improved gastric acid secretion. Therefore, a ghrelin receptor agonist represented by the working examples of the present invention enhances gastric acid secretion. The effect on enhancement of gastric acid secretion is shown to be useful in a variety of diseases in which low or no gastric acid secretion is involved.

Many ghrelin receptor agonists have been reported, but the effect on increasing gastric acid secretion has not been apparent to those skilled in the art so far, and according to this invention, a ghrelin receptor agonist enhances gastric acid secretion, which make it clear that a ghrelin receptor agonist is effective for alleviating or preventing various symptoms and diseases in which low or no gastric acid secretion is involved.

Compounds of the present invention for preventing or treating diseases involved in low or no gastric acid secretion include already known compounds having a ghrelin receptor agonist activity and also include compounds having a ghrelin receptor agonist activity which will be found hereafter.

Known examples of compounds having a ghrelin receptor agonistic activity are:

the compounds disclosed in WO97/024369, which are represented by capromorelin,
2-amino-N-[2-(3a-(R)-benzyl-2-methyl-3-oxo-2,3,3a,4,6,7-hexahydropyrazolo[4,3-c]pyridin-5-yl)-1-(R)-benzyloxymethyl-2-oxo-ethyl]isobutyramide and
2-amino-N-[1-(R)-(2,4-difluoro-benzyloxymethyl)-2-oxo-2-(3-oxo-3a-(R)-pyridin-2-ylmethyl-2-(2,2,2-trifluoro-ethyl)-2,3,3a,4,6,7-hexahydropyrazolo[4,3-c]pyridin-5-yl)-ethyl]-2-methyl-propionamide;

the compounds disclosed in WO99/58501 and WO2001/034593, which are represented by anamorelin,
2-amino-N-[(1R)-2-[(3R)-3-benzyl-3-(N,N′,N′-trimethylhydrazinocarbonyl)piperidin-1-yl]-1-(1H-indol-3-ylmethyl)-2-oxoethyl]-2-methylpropionamide, another name, 2-amino-N—((R)-1-((R)-3-benzyl-3-(1,2,2-trimethylhydrazinecarbonyl)piperidin-1-yl)-3-(1H-indol-3-yl)-1-oxopropan-2-yl)-2-methylpropanamide;

the compounds disclosed in WO2000/001726, which are represented by ST-1141, another name RC-1141,
(E)-N—((R)-3-([1,1′-biphenyl]-4-yl)-1-(((R)-1-(4-hydroxypiperidin-1-yl)-1-oxo-3-phen ylpropan-2-yl)(methyl)amino)-1-oxopropan-2-yl)-4-(1-aminocyclobutyl)-N-methylbut-2-enamide;

the compounds disclosed in WO2001/096300, which are represented by macimorelin,
2-amino-N—((R)-1-(((R)-1-formamido-2-(1H-indol-3-yl)ethyl)amino)-3-(1H-indol-3-yl)-1-oxopropan-2-yl)-2-methylpropanamide;

the compounds disclosed in WO2006/009674 and WO2011/041369, which are represented by ulimorelin,
(2R,5S,8R,11R)-5-cyclopropyl-11-(4-fluorobenzyl)-2,7,8-trimethyl-4,5,7,8,10,11,13,14,15,16-decahydro-2H-benzo[q][1,4,7,10,13]oxatetraazacyclooctadecine-6,9,12(3H)-trione;

the compounds disclosed in WO95/17423, which are represented by ipamorelin,
(S)-6-amino-2-((R)-2-((R)-2-((S)-2-(2-amino-2-methylpropanamido)-3-(1H-imidazol-5-yl)propanamido)-3-(naphthalen-2-yl)propanamido)-3-phenylpropanamido)hexanamide;
   and so on.

Compounds described in the literature cited above lead to all compounds described in claims of the above cited patents. Also, all of the above mentioned citations are incorporated in the description herein.

For oral administration of the compounds of the present invention, the molecular weight of lower than 800 is preferable taking gastrointestinal absorption into consideration.

Particularly, capromorelin, 2-amino-N-[1-(R)-(2,4-difluoro-benzyloxymethyl)-2-oxo-2-(3-oxo-3a-(R)-pyridin-2-yl methyl-2-(2,2,2-trifluoro-ethyl)-2,3,3a,4,6,7-hexahydropyrazolo[4,3-c]pyridin-5-yl)-ethyl]-2-methyl-propionamide; anamorelin, ST-1141, macimorelin, ulimorelin, ipamorelin are preferable. Compounds of the present invention include solvates, complexes, polymorphs, prodrugs, isomers and isotopically-labeled compounds thereof, as described below.

The gist of the present invention is as follows:

[1] A use of one or more selected from the group consisting of a compound of the formula (I), a racemic-diastereomeric mixture, and an optical isomer of the said compound, and a pharmaceutically-acceptable salt and a prodrug thereof, which may be abbreviated all together as the compound of the present invention, for the manufacture of a medicament for the treatment of achlorhydria in a human or an animal:

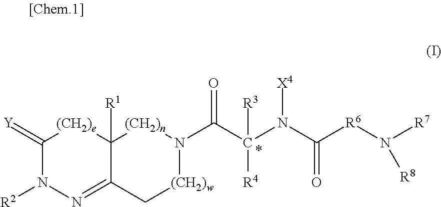

wherein

e is 0 or 1;

n and w are each independently 0, 1 or 2, provided that w and n cannot both be 0 at the same time;

Y is oxygen or sulfur;

R¹ is hydrogen, —CN, —(CH₂)_{q}N(X⁶)C(O)X⁶, —(CH₂)_{q}N(X⁶)C(O)(CH₂)ₜ-A¹, —(CH₂)N(X⁶)SO₂(CH₂)ₜ-A¹, —(CH₂)_{q}N(X⁶)SO₂X⁶, —(CH₂)N(X⁶)C(O)N(X⁶)(CH₂)ₜ-A¹, —(CH₂)_{q}N(X⁶)C(O)N(X⁶)(X⁶), —(CH₂)_{q}C(O)N(X⁶) (X⁶), —(CH₂)_{q}C(O)N(X⁶)(CH₂)ₜ-A¹, (CH₂)_{q}C(O)OX⁶, —(CH₂)_{q}C(O)O(CH₂)ₜ-A¹, —(CH₂)_{q}OX⁶, —(CH₂)_{q}OC(O)X⁶, —(CH₂)_{q}OC(O) (CH₂)ₜ-A¹, —(CH₂)_{q}OC(O)N(X⁶) (CH₂)ₜ-A¹, —(CH₂)_{q}OC(O)N(X⁶) (X⁶), —(CH₂)_{q}C(O)X⁶, —(CH₂)_{q}C(O)(CH₂)ₜ-A¹, —(CH₂)_{q}N(X⁶)C(O)OX⁶, —(CH₂)_{q}N(X⁶)SO₂N(X⁶)(X⁶), —(CH₂)_{q}S(O)ₘX⁶, —(CH₂)_{q}S(O)ₘ(CH₂)ₜ-A¹, —(C₁-C₁₀)alkyl, —(CH₂)ₜ-A¹, —(CH₂)_{q}(C₃-C₇)cycloalkyl, —(CH₂)_{q}—Y¹—(C₁-C₆)alkyl, —(CH₂)_{q}—Y¹—(CH₂)ₜ-A¹ or —(CH₂)_{q}—Y¹—(CH₂)ₜ—(C₃-C₇)cycloalkyl;

where the alkyl and cycloalkyl groups in the definition of R¹ are optionally substituted with (C₁-C₄)alkyl, hydroxyl, (C₁-C₄)alkoxy, carboxyl, —CONH₂,
—S(O)ₘ(C₁-C₆)alkyl, —CO₂(C₁-C₄)alkyl ester, 1H-tetrazol-5-yl or 1, 2 or 3 fluoro;

### Y¹ is O, S(O)ₘ, —C(O)NX⁶—, —CH═CH—, —C≡C—, —N(X⁶)C(O)—, —C(O)NX⁶—, —C(O)O—, —OC(O)N(X⁶)— or —OC(O)—;

q is 0, 1, 2, 3 or 4;
t is 0, 1, 2 or 3;
m is 0, 1 or 2;
said (CH₂)_{q} group and (CH₂)ₜ group may each be optionally substituted with hydroxyl, (C₁-C₄)alkoxy, carboxyl, —CONH₂, —S(O)ₘ—(C₁-C₆)alkyl,
—CO₂(C₁-C₄)alkyl ester, 1H-tetrazol-5-yl, 1, 2 or 3 fluoro, or 1 or 2 (C₁-C₄)alkyl;
R² is hydrogen, (C₁-C₈)alkyl, —(C₀-C₃)alkyl-(C₃-C₈)cycloalkyl, —(C₁-C₄)alkyl-A¹ or A¹;
where the alkyl groups and the cycloalkyl groups in the definition of R² are optionally substituted with hydroxyl, —C(O)OX⁶, —C(O)N(X⁶)(X⁶), —N(X⁶)(X⁶), —S(O)ₘ(C₁-C₆)alkyl, —C(O)A¹, —C(O)(X⁶), CF₃, CN or 1, 2 or 3 halogen;
R³ is A¹, (C₁-C₁₀)alkyl, —(C₁-C₆)alkyl-Al, —(C₁-C₆)alkyl-(C₃-C₇)cycloalkyl, —(C₁-C₅)alkyl-X¹—(C₁-C₅)alkyl, —(C₁-C₅)alkyl-X¹—(C₀-C₅)alkyl-A¹ or —(C₁-C₅)alkyl-X¹—(C₁-C₅)alkyl-(C₃-C₇)cycloalkyl;
where the alkyl groups in the definition of R³ are optionally substituted with, —S(O)ₘ(C₁-C₆)alkyl, —C(O)OX³, 1, 2, 3, 4 or 5 halogens, or 1, 2 or 3 OX³;
X¹ is O, S(O)ₘ, —N(X²)C(O)—, —C(O)N(X²)—, —OC(O)—, —C(O)O—, —CX²═CX²—, —N(X²)C(O)O—, —OC(O)N(X²)— or —C≡C—;
R⁴ is hydrogen, (C₁-C₆)alkyl or (C₃-C₇)cycloalkyl, or R⁴ is taken together with R³ and the carbon atom to which they are attached and form (C₅-C₇)cycloalkyl, (C₅-C₇)cycloalkenyl, a partially saturated or fully saturated 4- to 8-membered ring having 1 to 4 heteroatoms independently selected from the group consisting of oxygen, sulfur and nitrogen, or R⁴ is a bicyclic ring system consisting of a partially saturated or fully saturated 5- or 6-membered ring, fused to a partially saturated, fully unsaturated or fully saturated 5- or 6-membered ring, optionally having 1 to 4 heteroatoms independently selected from the group consisting of nitrogen, sulfur and oxygen;
X⁴ is hydrogen or (C₁-C₆)alkyl or X⁴ is taken together with R⁴ and the nitrogen atom to which X⁴ is attached and the carbon atom to which R⁴ is attached and form a five to seven membered ring;
R⁶ is a bond or

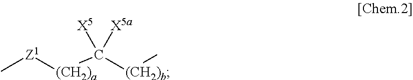

where a and b are independently 0, 1, 2 or 3;

X⁵ and X^{5a} are each independently selected from the group consisting of hydrogen, trifluoromethyl, A¹ and optionally substituted (C₁-C₆)alkyl;

the optionally substituted (C₁-C₆)alkyl in the definition of X⁵ and X^{5a} is optionally substituted with a substituent selected from the group consisting of A¹, OX², —S(O)ₘ(C₁-C₆)alkyl, —C(O)OX², (C₃-C₇)cycloalkyl, —N(X²)(X²) and —C(O)N(X²)(X²);

in which the carbon bearing X⁵ or X^{5a} forms one or two alkylene bridges with the nitrogen atom bearing R⁷ and R⁸ wherein each alkylene bridge contains 1 to 5 carbon atoms, provided that when one alkylene bridge is formed then X⁵ or X^{5a} but not both may be on the carbon atom and R⁷ or R⁸ but not both may be on the nitrogen atom and further provided that when two alkylene bridges are formed then X⁵ and X^{5a} cannot be on the carbon atom and R⁷ and R⁸ cannot be on the nitrogen atom;

or X⁵ is taken together with X^{5a} and the carbon atom to which they are attached and form a partially saturated or fully saturated 3- to 7-membered ring, or a partially saturated or fully saturated 4- to 8-membered ring having 1 to 4 heteroatoms independently selected from the group consisting of oxygen, sulfur and nitrogen,

or X⁵ is taken together with X^{5a} and the carbon atom to which they are attached and form a bicyclic ring system consisting of a partially saturated or fully saturated 5- or 6-membered ring, optionally having 1 or 2 heteroatoms independently selected from the group consisting of nitrogen, sulfur and oxygen, fused to a partially saturated, fully saturated or fully unsaturated 5- or 6-membered ring, optionally having 1 to 4 heteroatoms independently selected from the group consisting of nitrogen, sulfur and oxygen;

Z¹ is a bond, O or N—X², provided that when a and b are both 0 then Z¹ is not N—X² or O;

R⁷ and R⁸ are independently hydrogen or optionally substituted (C₁-C₆)alkyl;

where the optionally substituted (C₁-C₆)alkyl in the definition of R⁷ and R⁸ is optionally independently substituted with A¹, —C(O)O—(C₁-C₆)alkyl, —S(O)ₘ(C₁-C₆)alkyl, 1 to 5 halogens, 1 to 3 hydroxy groups, 1 to 3 —O—C(O)(C₁-C₁₀)alkyl groups or 1 to 3 (C₁-C₆)alkoxy groups; or

R⁷ and R⁸ can be taken together to form —(CH₂)ᵣ-L-(CH₂)ᵣ—;
where L is C(X²)(X²), S(O)ₘ or N(X²);
A¹ for each occurrence is independently (C₅-C₇)cycloalkenyl, phenyl or substituent formed by eliminating hydrogen atom from a partially saturated, fully saturated or fully unsaturated 4- to 8-membered ring optionally having 1 to 4 heteroatoms independently selected from the group consisting of oxygen, sulfur and nitrogen, a bicyclic ring system consisting of a partially saturated, fully unsaturated or fully saturated 5- or 6-membered ring, optionally having 1 to 4 heteroatoms independently selected from the group consisting of nitrogen, sulfur and oxygen, fused to a partially saturated, fully saturated or fully unsaturated 5- or 6-membered ring, optionally having 1 to 4 heteroatoms independently selected from the group consisting of nitrogen, sulfur and oxygen;
A¹ for each occurrence is independently optionally substituted, in one or optionally both rings if A¹ is a bicyclic ring system, with up to three substituents, each substituent independently selected from the group consisting of F, Cl, Br, I, OCF₃, OCF₂H, CF₃, CH₃, OCH₃, —OX⁶, —C(O)N(X⁶)(X⁶), —C(O)OX⁶, oxo, (C₁-C₆)alkyl, nitro, cyano, benzyl, —S(O)ₘ(C₁-C₆)alkyl, 1H-tetrazol-5-yl, phenyl, phenoxy, phenylalkyloxy, halophenyl, methylenedioxy, —N(X⁶)(X⁶), —N(X⁶)C(O)(X⁶), —SO₂N(X⁶)(X⁶), —N(X⁶)SO₂-phenyl, —N(X⁶)SO₂X⁶, —CONX¹¹X¹², —SO₂NX¹¹X¹², —NX⁶SO₂X¹², —NX⁶CONX¹¹X¹², —NX⁶SO₂NX¹¹X¹², —NX⁶C(O)X¹², imidazolyl, thiazolyl or tetrazolyl, provided that if A¹ is optionally substituted with methylenedioxy then it can only be substituted with one methylenedioxy;
where X¹¹ is hydrogen or optionally substituted (C₁-C₆)alkyl;
the optionally substituted (C₁-C₆)alkyl defined for X¹¹ is optionally independently substituted with phenyl, phenoxy, (C₁-C₆)alkoxycarbonyl, —S(O)ₘ(C₁-C₆)alkyl, 1 to 5 halogens, 1 to 3 hydroxy, 1 to 3 (C₁-C₁₀)alkanoyloxy or 1 to 3 (C₁-C₆)alkoxy;
X¹² is hydrogen, (C₁-C₆)alkyl, phenyl, thiazolyl, imidazolyl, furyl or thienyl, provided that when X¹² is not hydrogen, X¹² is optionally substituted with one to three substituents independently selected from the group consisting of Cl, F, CH₃, OCH₃, OCF₃ and CF₃;
or X¹¹ and X¹² are taken together to form —(CH₂)ᵣ-L¹-(CH₂)ᵣ—;
where L¹ is C(X²)(X²), O, S(O)ₘ or N(X²);
r for each occurrence is independently 1, 2 or 3;
X² for each occurrence is independently hydrogen, optionally substituted (C₁-C₆)alkyl, or optionally substituted (C₃-C₇)cycloalkyl, where the optionally substituted (C₁-C₆)alkyl and optionally substituted (C₃-C₇)cycloalkyl in the definition of X² are optionally independently substituted with —S(O)ₘ(C₁-C₆)alkyl, —C(O)OX³, 1 to 5 halogens or 1 to 3 OX³;
X³ for each occurrence is independently hydrogen or (C₁-C₆)alkyl;
X⁶ is independently hydrogen, optionally substituted (C₁-C₆)alkyl, (C₂-C₆)halogenated alkyl, optionally substituted (C₃-C₇)cycloalkyl, (C₃-C₇)-halogenatedcycloalkyl, where optionally substituted (C₁-C₆)alkyl and optionally substituted (C₃-C₇)cycloalkyl in the definition of X⁶ is optionally independently substituted by 1 or 2 (C₁-C₄)alkyl, hydroxyl, (C₁-C₄)alkoxy, carboxyl, CONH₂, —S(O)ₘ(C₁-C₆)alkyl, carboxylate, (C₁-C₄)alkyl carboxy ester, or 1H-tetrazol-5-yl; or
when there are two X⁶ groups on one atom and both X⁶ are independently (C₁-C₆)alkyl, the two (C₁-C₆)alkyl groups may be optionally joined and, together with the atom to which the two X⁶ groups are attached, form a 4- to 9-membered ring optionally having oxygen, sulfur or NX⁷;
X⁷ is hydrogen or (C₁-C₆)alkyl optionally substituted with hydroxyl; and m for each occurrence is independently 0, 1 or 2;
with the proviso that:
X⁶ and X¹² cannot be hydrogen when it is attached to C(O) or SO₂ in the form C(O)X⁶, C(O)X¹², SO₂X⁶ or SO₂X¹²; and
when R⁶ is a bond then L is N(X²) and each r in the definition —(CH₂)ᵣ-L-(CH₂)ᵣ— is independently 2 or 3;

[2] A use of one or more selected from the group consisting of a compound of the formula (II), a racemic-diastereomeric mixture, and an optical isomer of the said compound, and a pharmaceutically-acceptable salt and a prodrug thereof for the manufacture of a medicament for the treatment of achlorhydria in human or animal:

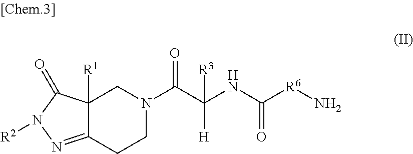

wherein

R¹ is —(C₁-C₃)alkyl-phenyl, —(C₁-C₃)alkyl-pyridyl, —(C₁-C₃)alkyl-quinolyl or —(C₁-C₃)alkyl-thiazolyl, where the phenyl in R¹ is optionally substituted with one or two substituents selected from the group consisting of halo, CF₃, CH₃ and phenyl;

R² is —(C₁-C₄)alkyl or —(C₁-C₄)alkyl-CF₃;

R³ is —(C₁-C₄)alkylindolyl, —(C₁-C₄)alkylphenyl, —(C₁-C₄)alkyl-O—(C₁-C₄)alkyl-Ar, —(C₁-C₄)alkyl-S—(C₁-C₄)alkyl-Ar, where Ar is phenyl, thienyl, thiazolyl, pyridyl, pyrimidinyl or benzisoxazolyl, the said Ar is optionally substituted with one or two substituents selected from the group consisting of halo, OCF₃, CF₃ and CH₃; and
R⁶ is —C(X⁵)(X⁵), where X⁵ is —(C₁-C₆)alkyl;

[3] The use according to [1] or [2], wherein the compound is selected from the group consisting of the following compounds:
2-amino-N-[1-(3a-(R,S)-benzyl-2-methyl-3-oxo-2,3,3a,4,6,7-hexahydropyrazolo[4,3-c]pyridine-5-carbonyl)-4-phenyl(R)-butyl]isobutyramide;
2-amino-N-[1-(3a-(R)-benzyl-2-methyl-3-oxo-2,3,3a,4,6,7-hexahydropyrazolo[4,3-c]pyridine-5-carbonyl)-4-phenyl(R)-butyl]isobutyramide;
2-amino-N-[1-(3a-(S)-benzyl-2-methyl-3-oxo-2,3,3a,4,6,7-hexahydropyrazolo[4,3-c]pyridine-5-carbonyl)-4-phenyl(R)-butyl]isobutyramide;
2-amino-N-[2-(3a-(R,S)-benzyl-2-methyl-3-oxo-2,3,3a,4,6,7-hexahydropyrazolo[4,3-c]pyridin-5-yl)-1-(R)-(1H-indol-3-ylmethyl)-2-oxo-ethyl]isobutyramide;
2-amino-N-[2-(3a-(R)-benzyl-2-methyl-3-oxo-2,3,3a,4,6,7-hexahydropyrazolo[4,3-c]pyridin-5-yl)-1-(R)-(1H-indol-3-ylmethyl)-2-oxo-ethyl]isobutyramide;
2-amino-N-[2-(3a-(S)-benzyl-2-methyl-3-oxo-2,3,3a,4,6,7-hexahydropyrazolo[4,3-c]pyridin-5-yl)-1-(R)-(1H-indol-3-ylmethyl)-2-oxo-ethyl]isobutyramide;
2-amino-N-[2-(3a-(R,S)-benzyl-2-ethyl-3-oxo-2,3,3a,4,6,7-hexahydropyrazolo[4,3-c]pyridin-5-yl)-1-(R)-(1H-indol-3-ylmethyl)-2-oxo-ethyl]isobutyramide;
2-amino-N-[2-(3a-(R)-benzyl-2-ethyl-3-oxo-2,3,3a,4,6,7-hexahydropyrazolo[4,3-c]pyridin-5-yl)-1-(R)-(1H-indol-3-ylmethyl)-2-oxo-ethyl]isobutyramide;
2-amino-N-[2-(3a-(S)-benzyl-2-ethyl-3-oxo-2,3,3a,4,6,7-hexahydropyrazolo[4,3-c]pyridin-5-yl)-1-(R)-(1H-indol-3-ylmethyl)-2-oxo-ethyl]isobutyramide;
2-amino-N-[2-(3a-(R,S)-(4-fluoro-benzyl)-2-methyl-3-oxo-2,3,3a,4,6,7-hexahydropyrazolo[4,3-c]pyridin-5-yl)-1-(R)-(1H-indol-3-ylmethyl)-2-oxo-ethyl]isobutyramide;
2-amino-N-[2-(3a-(R)-(4-fluoro-benzyl)-2-methyl-3-oxo-2,3,3a,4,6,7-hexahydropyrazolo[4,3-c]pyridin-5-yl)-1-(R)-(1H-indol-3-ylmethyl)-2-oxo-ethyl]isobutyramide;
2-amino-N-[2-(3a-(S)-(4-fluoro-benzyl)-2-methyl-3-oxo-2,3,3a,4,6,7-hexahydropyrazolo[4,3-c]pyridin-5-yl)-1-(R)-(1H-indol-3-ylmethyl)-2-oxo-ethyl]isobutyramide;
2-amino-N-[2-(3a-(R,S)-benzyl-2-methyl-3-oxo-2,3,3a,4,6,7-hexahydropyrazolo[4,3-c]pyridin-5-yl)-1-(R)-benzyloxymethyl-2-oxo-ethyl]isobutyramide;
2-amino-N-[2-(3a-(R)-benzyl-2-methyl-3-oxo-2,3,3a,4,6,7-hexahydropyrazolo[4,3-c]pyridin-5-yl)-1-(R)-benzyloxymethyl-2-oxo-ethyl]isobutyramide;
2-amino-N-[2-(3a-(S)-benzyl-2-methyl-3-oxo-2,3,3a,4,6,7-hexahydropyrazolo[4,3-c]pyridin-5-yl)-1-(R)-benzyloxymethyl-2-oxo-ethyl]isobutyramide;
2-amino-N-[2-(3a-(R,S)-benzyl-2-ethyl-3-oxo-2,3,3a,4,6,7-hexahydropyrazolo[4,3-c]pyridin-5-yl)-1-(R)-benzyloxymethyl-2-oxo-ethyl]isobutyramide;
2-amino-N-[2-(3a-(R)-benzyl-2-ethyl-3-oxo-2,3,3a,4,6,7-hexahydropyrazolo[4,3-c]pyridin-5-yl)-1-(R)-benzyloxymethyl-2-oxo-ethyl]isobutyramide;
2-amino-N-[2-(3a-(S)-benzyl-2-ethyl-3-oxo-2,3,3a,4,6,7-hexahydropyrazolo[4,3-c]pyridin-5-yl)-1-(R)-benzyloxymethyl-2-oxo-ethyl]isobutyramide;
2-amino-N-[2-(3a-(R,S)-benzyl-3-oxo-2-(2,2,2-trifluoro-ethyl)-2,3,3a,4,6,7-hexahydro pyrazolo[4,3-c]pyridin-5-yl)-1-(R)-benzyloxymethyl-2-oxo-ethyl]isobutyramide;
2-amino-N-[2-(3a-(R)-benzyl-3-oxo-2-(2,2,2-trifluoro-ethyl)-2,3,3a,4,6,7-hexahydropyrazolo[4,3-c]pyridin-5-yl)-1-(R)-benzyloxymethyl-2-oxo-ethyl]isobutyramide;
2-amino-N-[2-(3a-(S)-benzyl-3-oxo-2-(2,2,2-trifluoro-ethyl)-2,3,3a,4,6,7-hexahydropyrazolo[4,3-c]pyridin-5-yl)-1-(R)-benzyloxymethyl-2-oxo-ethyl]isobutyramide;
2-amino-N-[1-(R)-benzyloxymethyl-2-(3a-(R,S)-(4-fluoro-benzyl)-2-methyl-3-oxo-2,3,3a,4,6,7-hexahydropyrazolo[4,3-c]pyridin-5-yl)-2-oxo-ethyl]isobutyramide;
2-amino-N-[1-(R)-benzyloxymethyl-2-(3a-(R)-(4-fluoro-benzyl)-2-methyl-3-oxo-2,3,3a,4,6,7-hexahydropyrazolo[4,3-c]pyridin-5-yl)-2-oxo-ethyl]isobutyramide;
2-amino-N-[1-(R)-benzyloxymethyl-2-(3a-(S)-(4-fluoro-benzyl)-2-methyl-3-oxo-2,3,3a,4,6,7-hexahydropyrazolo[4,3-c]pyridin-5-yl)-2-oxo-ethyl]isobutyramide;
2-amino-N-[2-(3a-(R,S)-benzyl-2-tert-butyl-3-oxo-2,3,3a,4,6,7-hexahydropyrazolo[4,3-c]pyridin-5-yl)-1-(R)-benzyloxymethyl-2-oxo-ethyl]isobutyramide;
2-amino-N-[2-(3a-(R)-benzyl-2-tert-butyl-3-oxo-2,3,3a,4,6,7-hexahydropyrazolo[4,3-c]pyridin-5-yl)-1-(R)-benzyloxymethyl-2-oxo-ethyl]isobutyramide;
2-amino-N-[2-(3a-(S)-benzyl-2-tert-butyl-3-oxo-2,3,3a,4,6,7-hexahydropyrazolo[4,3-c]pyridin-5-yl)-1-(R)-benzyloxymethyl-2-oxo-ethyl]isobutyramide;
2-amino-N-[2-(3a-(R,S)-benzyl-3-oxo-2,3,3a,4,6,7-hexahydropyrazolo[4,3-c]pyridin-5-yl)-1-(R)-benzyloxymethyl-2-oxo-ethyl]isobutyramide;
2-amino-N-[2-(3a-(R)-benzyl-3-oxo-2,3,3a,4,6,7-hexahydropyrazolo[4,3-c]pyridin-5-y 1)-1-(R)-benzyloxymethyl-2-oxo-ethyl]isobutyramide;
2-amino-N-[2-(3a-(S)-benzyl-3-oxo-2,3,3a,4,6,7-hexahydropyrazolo[4,3-c]pyridin-5-yl)-1-(R)-benzyloxymethyl-2-oxo-ethyl]isobutyramide;
2-amino-N-[1-(R)-benzyloxymethyl-2-(2-methyl-3-oxo-3a-(R,S)-pyridin-2-ylmethyl-2,3,3a,4,6,7-hexahydropyrazolo[4,3-c]pyridin-5-yl)-2-oxo-ethyl]-2-methyl-propionamide;
2-amino-N-[1-(R)-benzyloxymethyl-2-(2-methyl-3-oxo-3a-(R)-pyridin-2-ylmethyl-2,3,3a,4,6,7-hexahydropyrazolo[4,3-c]pyridin-5-yl)-2-oxo-ethyl]-2-methyl-propionamide;
2-amino-N-[1-(R)-benzyloxymethyl-2-(2-methyl-3-oxo-3a-(S)-pyridin-2-ylmethyl-2,3,3a,4,6,7-hexahydropyrazolo[4,3-c]pyridin-5-yl)-2-oxo-ethyl]-2-methyl-propionamide;
2-amino-N-[1-(R)-(3-chloro-benzyloxymethyl)-2-oxo-2-(3-oxo-3a-(R,S)-pyridin-2-yl methyl-2-(2,2,2-trifluoro-ethyl)-2,3,3a,4,6,7-hexahydropyrazolo[4,3-c]pyridin-5-yl)-ethyl]-2-methyl-propionamide;
2-amino-N-[1-(R)-(3-chloro-benzyloxymethyl)-2-oxo-2-(3-oxo-3a-(R)-pyridin-2-ylmethyl-2-(2,2,2-trifluoro-ethyl)-2,3,3a,4,6,7-hexahydropyrazolo[4,3-c]pyridin-5-yl)-ethyl]-2-methyl-propionamide;
2-amino-N-[1-(R)-(3-chloro-benzyloxymethyl)-2-oxo-2-(3-oxo-3a-(S)-pyridin-2-ylmethyl-2-(2,2,2-trifluoro-ethyl)-2,3,3a,4,6,7-hexahydropyrazolo[4,3-c]pyridin-5-yl)-ethyl]-2-methyl-propionamide;
2-amino-N-[1-(R)-(4-chloro-benzyloxymethyl)-2-oxo-2-(3-oxo-3a-(R,S)-pyridin-2-yl methyl-2-(2,2,2-trifluoro-ethyl)-2,3,3a,4,6,7-hexahydropyrazolo[4,3-c]pyridin-5-yl)-ethyl]-2-methyl-propionamide;
2-amino-N-[1-(R)-(4-chloro-benzyloxymethyl)-2-oxo-2-(3-oxo-3a-(R)-pyridin-2-ylmethyl-2-(2,2,2-trifluoro-ethyl)-2,3,3a,4,6,7-hexahydropyrazolo[4,3-c]pyridin-5-yl)-ethyl]-2-methyl-propionamide;
2-amino-N-[1-(R)-(4-chloro-benzyloxymethyl)-2-oxo-2-(3-oxo-3a-(S)-pyridin-2-ylmethyl-2-(2,2,2-trifluoro-ethyl)-2,3,3a,4,6,7-hexahydropyrazolo[4,3-c]pyridin-5-yl)-ethyl]-2-methyl-propionamide;
2-amino-N-[1-(R)-(2,4-dichloro-benzyloxymethyl)-2-oxo-2-(3-oxo-3a-(R,S)-pyridin-2-ylmethyl-2-(2,2,2-trifluoro-ethyl)-2,3,3a,4,6,7-hexahydropyrazolo[4,3-c]pyridin-5-yl)-ethyl]-2-methyl-propionamide;
2-amino-N-[1-(R)-(2,4-dichloro-benzyloxymethyl)-2-oxo-2-(3-oxo-3a-(R)-pyridin-2-ylmethyl-2-(2,2,2-trifluoro-ethyl)-2,3,3a,4,6,7-hexahydropyrazolo[4,3-c]pyridin-5-yl)-ethyl]-2-methyl-propionamide;
2-amino-N-[1-(R)-(2,4-dichloro-benzyloxymethyl)-2-oxo-2-(3-oxo-3a-(S)-pyridin-2-yl methyl-2-(2,2,2-trifluoro-ethyl)-2,3,3a,4,6,7-hexahydropyrazolo[4,3-c]pyridin-5-yl)-ethyl]-2-methyl-propionamide;
2-amino-N-[1-(R)-(4-chloro-thiophen-2-ylmethoxymethyl)-2-oxo-2-(3-oxo-3a-(R,S)-pyridin-2-ylmethyl-2-(2,2,2-trifluoro-ethyl)-2,3,3a,4,5,7-hexahydropyrazolo[3,4-c]pyridin-6-yl)-ethyl]-2-methyl-propionamide;
2-amino-N-[1-(R)-(4-chloro-thiophen-2-ylmethoxymethyl)-2-oxo-2-(3-oxo-3a-(R)-pyridin-2-ylmethyl-2-(2,2,2-trifluoro-ethyl)-2,3,3a,4,5,7-hexahydropyrazolo[3,4-c]pyridin-6-yl)-ethyl]-2-methyl-propionamide;
2-amino-N-[1-(R)-(4-chloro-thiophen-2-ylmethoxymethyl)-2-oxo-2-(3-oxo-3a-(S)-pyridin-2-ylmethyl-2-(2,2,2-trifluoro-ethyl)-2,3,3a,4,5,7-hexahydropyrazolo[3,4-c]pyridin-6-yl)-ethyl]-2-methyl-propionamide;
2-amino-N-[1-(R)-(2,4-difluoro-benzyloxymethyl)-2-oxo-2-(3-oxo-3a-(R,S)-pyridin-2-ylmethyl-2-(2,2,2-trifluoro-ethyl)-2,3,3a,4,6,7-hexahydropyrazolo[4,3-c]pyridin-5-yl)-ethyl]-2-methyl-propionamide;
2-amino-N-[1-(R)-(2,4-difluoro-benzyloxymethyl)-2-oxo-2-(3-oxo-3a-(R)-pyridin-2-ylmethyl-2-(2,2,2-trifluoro-ethyl)-2,3,3a,4,6,7-hexahydropyrazolo[4,3-c]pyridin-5-yl)-ethyl]-2-methyl-propionamide;
2-amino-N-[1-(R)-(2,4-difluoro-benzyloxymethyl)-2-oxo-2-(3-oxo-3a-(S)-pyridin-2-yl methyl-2-(2,2,2-trifluoro-ethyl)-2,3,3a,4,6,7-hexahydropyrazolo[4,3-c]pyridin-5-yl)-ethyl]-2-methyl-propionamide;
2-amino-N-[2-(3a-(R,S)-benzyl-2-methyl-3-oxo-2,3,3a,4,6,7-hexahydropyrazolo[4,3-c]pyridin-5-yl)-1-(R)-(3,4-difluoro-benzyloxymethyl)-2-oxo-ethyl]-2-methyl-propionamide;
2-amino-N-[2-(3a-(R)-benzyl-2-methyl-3-oxo-2,3,3a,4,6,7-hexahydropyrazolo[4,3-c]pyridin-5-yl)-1-(R)-(3,4-difluoro-benzyloxymethyl)-2-oxo-ethyl]-2-methyl-propionamide;
2-amino-N-[2-(3a-(S)-benzyl-2-methyl-3-oxo-2,3,3a,4,6,7-hexahydropyrazolo[4,3-c]pyridin-5-yl)-1-(R)-(3,4-difluoro-benzyloxymethyl)-2-oxo-ethyl]-2-methyl-propionamide; and
a pharmaceutically acceptable salt thereof;

[4] The use according to any one of [1] to [3], wherein the compound is selected from the group consisting of the following compounds:
2-amino-N-[2-(3a-(R)-benzyl-2-methyl-3-oxo-2,3,3a,4,6,7-hexahydropyrazolo[4,3-c]pyridin-5-yl)-1-(R)-benzyloxymethyl-2-oxo-ethyl]isobutyramide;
2-amino-N-[1-(R)-(2,4-difluoro-benzyloxymethyl)-2-oxo-2-(3-oxo-3a-(R)-pyridin-2-ylmethyl-2-(2,2,2-trifluoro-ethyl)-2,3,3a,4,6,7-hexahydropyrazolo[4,3-c]pyridin-5-yl)-ethyl]-2-methyl-propionamide; and
a pharmaceutically acceptable salt thereof;

[5] A use of one or more selected from the group consisting of a compound of the formula (III), a racemic-diastereomeric mixture, and an optical isomer of the said compound, and a pharmaceutically-acceptable salt and a prodrug thereof for the manufacture of a medicament for the treatment of achlorhydria in a human or an animal:

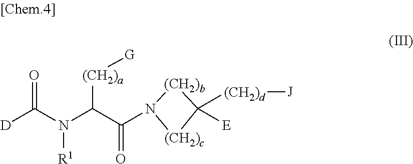

wherein
R¹ is hydrogen, or C₁₋₆-alkyl optionally substituted with one or more aryl or hetaryl;
a and d are independently of each other 0, 1, 2 or 3;
b and c are independently of each other 0, 1, 2, 3, 4 or 5, provided that b+c is 3, 4 or 5,

### D is

R²—NH—(CR³R⁴)ₑ—(CH₂)_{f}-M-(CHR⁵)_{g}—(CH₂)ₕ—

wherein R², R³, R⁴ and R⁵ are independently hydrogen or C₁₋₆-alkyl optionally substituted with one or more halogen, amino, hydroxyl, aryl or hetaryl; or

R² and R³ or R² and R⁴ or R³ and R⁴ may optionally form —(CH₂)ᵢ—U—(CH₂)ⱼ—, wherein i and j are independently 1 or 2 and U is —O—, —S— or a bond;

h and f are independently 0, 1, 2, or 3;

g and e are independently 0 or 1;

M is a bond, —CR⁶═CR⁷—, arylene, hetarylene, —O— or —S—;

R⁶ and R⁷ are independently hydrogen, or C₁₋₆-alkyl optionally substituted with one or more aryl or hetaryl;

G is —O—(CH₂)ₖ—R⁸,

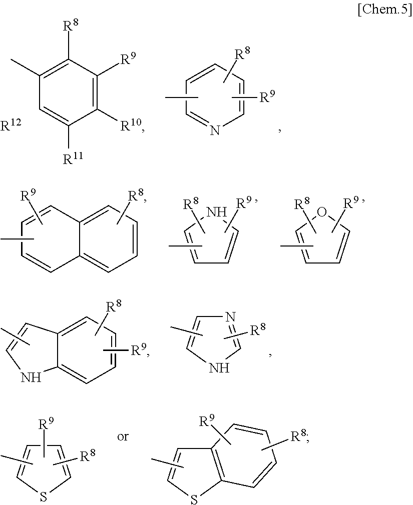

J is —O—(CH₂)₁R¹³,

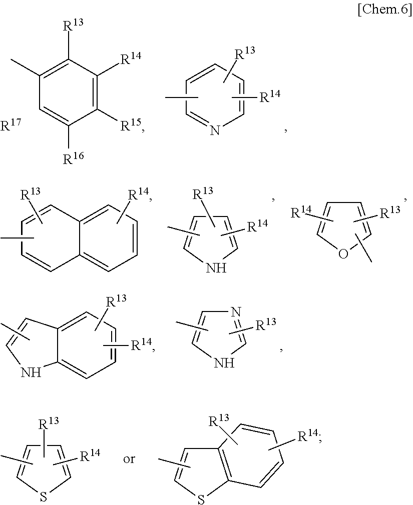

wherein R⁸, R⁹, R¹⁰, R¹¹, R¹², R¹³, R¹⁴, R¹⁵, R¹⁶ and R¹⁷ independently of each other are hydrogen, halogen, aryl, hetaryl, C₁₋₆-alkyl or C₁₋₆-alkoxy;

k and I are independently 0, 1 or 2;
E is —CONR¹⁸R¹⁹, —COOR¹⁹, —(CH₂)ₘ—NR¹⁸SO₂R²⁰, —(CH₂)ₘ—NR¹⁸COR²⁰, —(CH₂)ₘ—OR¹⁹, —(CH₁)—OCOR²⁰, —CH(R¹⁸)R¹⁹, —(CH₂)ₘ—NR¹⁸—CS—NR¹⁹R²¹ or
—(CH₂)ₘ—NR¹⁸—CO—NR¹⁹R²¹; or

### E is —CONR²²NR²³R²⁴,

wherein R²² is hydrogen, C₁₋₆-alkyl optionally substituted with one or more aryl or hetaryl, or aryl or hetaryl optionally substituted with one or more C₁₋₆-alkyl; R²³ is C₁₋₆-alkyl optionally substituted with one or more aryl or hetaryl, or C₁₋₇-acyl; and R²⁴ is hydrogen, C₁₋₆-alkyl optionally substituted with one or more aryl or hetaryl; or aryl or hetaryl optionally substituted with one or more C₁₋₆-alkyl; or
R²² and R²³ together with the nitrogen atoms to which they are attached may form a heterocyclic system optionally substituted with one or more C₁₋₆-alkyl, halogen, amino, hydroxyl, aryl or hetaryl; or
R²² and R²⁴ together with the nitrogen atoms to which they are attached may form a heterocyclic system optionally substituted with one or more C₁₋₆-alkyl, halogen, amino, hydroxyl, aryl or hetaryl; or
R²³ and R²⁴ together with the nitrogen atom to which they are attached may form a heterocyclic system optionally substituted with one or more C₁₋₆-alkyl, halogen, amino, hydroxyl, aryl or hetaryl;
wherein m is 0, 1, 2 or 3,
R¹⁸, R¹⁹ and R²¹ independently are hydrogen or C₁₋₆-alkyl optionally substituted with halogen, —N(R²⁵)R²⁶, wherein R²⁵ and R²⁶ are independently hydrogen or C₁₋₆-alkyl;
hydroxyl, C₁₋₆-alkoxy, C₁₋₆-alkoxycarbonyl, C₁₋₆-alkylcarbonyloxy or aryl;

or R¹⁹ is

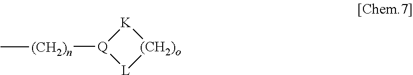

wherein

### Q is —CH< or —N<,

K and L are independently —CH₂, —CO—, —O—, —S—, —NR²⁷— or a bond,
where R²⁷ is hydrogen or C₁₋₆-alkyl;
n and o are independently 0, 1, 2, 3 or 4;
R²⁰ is C₁₋₆-alkyl, aryl or hetaryl;
or a pharmaceutically acceptable salt thereof;
with the proviso that
if M is a bond then E is —CONR²²NR²³R²⁴;

[6] The use according to [5], wherein the compound is following formula (IV):

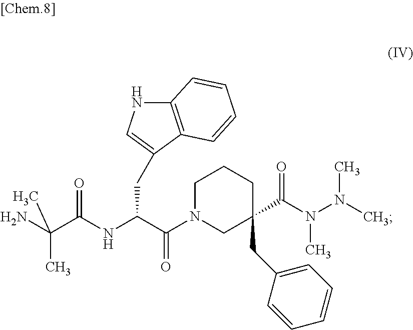 [SMILES: CN(C)N(C)C(=O)[C@@]1(Cc2ccccc2)CCCN(C(=O)[C@@H](Cc2c[nH]c3ccccc23)NC(=O)C(C)(C)N)C1]

[7] A use of one or more selected from the group consisting of a compound of the formula (V), a racemic-diastereomeric mixture, and an optical isomer of the said compound, and a pharmaceutically-acceptable salt and a prodrug thereof for the manufacture of a medicament for the treatment of achlorhydria in a human or an animal:

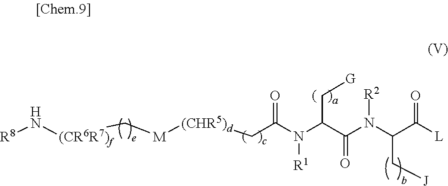

wherein

R¹ is hydrogen or C₁₋₆-alkyl;

R² is hydrogen or C₁₋₆-alkyl;

L is

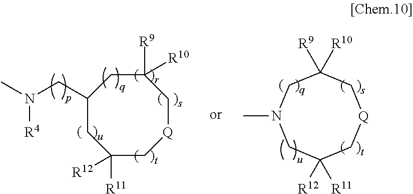

wherein R⁴ is hydrogen or C₁₋₆-alkyl;

p is 0 or 1;

q, s, t, u are independently from each other 0, 1, 2, 3 or 4;

r is 0 or 1;

the sum q+r+s+t+u is 0, 1, 2, 3, or 4;

R⁹, R¹⁰, R¹¹, and R¹² are independently from each other hydrogen or C₁₋₆-alkyl;

Q is >N—R¹³ or

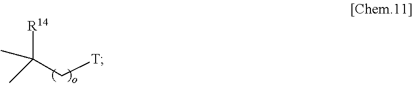

wherein o is 0, 1 or 2;

T is —N(R¹⁵)(R¹⁶) or hydroxyl;

R¹³, R¹⁵, and R¹⁶ are independently from each other hydrogen or C₁₋₆-alkyl;

R¹⁴ is hydrogen, aryl or hetaryl;

G is —O—(CH₂)ₖ—R¹⁷,

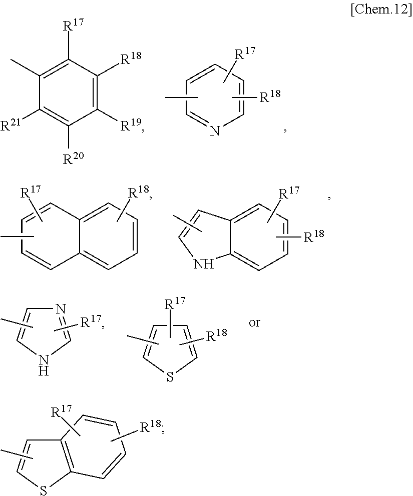

wherein R¹⁷, R¹⁸, R¹⁹, R²⁰ and R²¹ independently from each other are hydrogen, halogen, aryl, hetaryl, C₁₋₆-alkyl or C₁₋₆-alkoxy;

k is 0, 1 or 2;

J is —O—(CH₂)₁R²²,

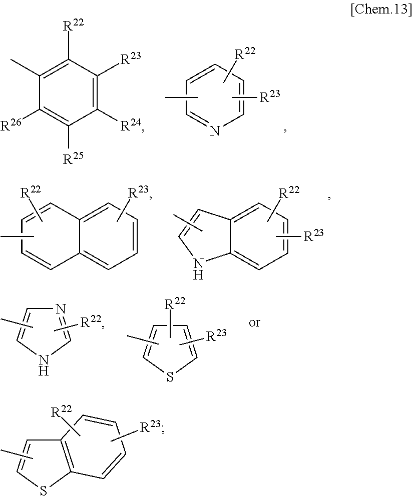

wherein R²², R²³, R²⁴, R²⁵ and R²⁶ independently from each other are hydrogen, halogen, aryl, hetaryl, C₁₋₆-alkyl or C₁₋₆-alkoxy;

I is 0, 1 or 2;

a is 0, 1, or 2;

b is 0, 1, or 2;

c is 0, 1, or 2;

d is 0 or 1;
e is 0, 1, 2, or 3;
f is 0 or 1;
R⁵ is hydrogen or C₁₋₆-alkyl optionally substituted with one or more hydroxyl, aryl or hetaryl;
R⁶ and R⁷ are independently from each other hydrogen or C₁₋₆-alkyl, optionally substituted with one or more halogen, amino, hydroxyl, aryl, or hetaryl;
R⁸ is hydrogen or C₁₋₆-alkyl, optionally substituted with one or more halogen, amino, hydroxyl, aryl, or hetaryl;
R⁸ and R⁷ or R⁶ and R⁸ or R⁷ and R⁸ may optionally form —(CH₂)ᵢ—U—(CH₂)ⱼ—, wherein i and j independently from each other are 1, 2 or 3 and U is —O—, —S—, or a bond;
M is arylene, hetarylene, —O—, —S— or —CR²⁷═CR²⁸—;
R²⁷ and R²⁸ are independently from each other hydrogen or C₁₋₆-alkyl, optionally substituted with one or more aryl or hetaryl;

[8] The use according to [7], wherein the compound is following formula (VI):

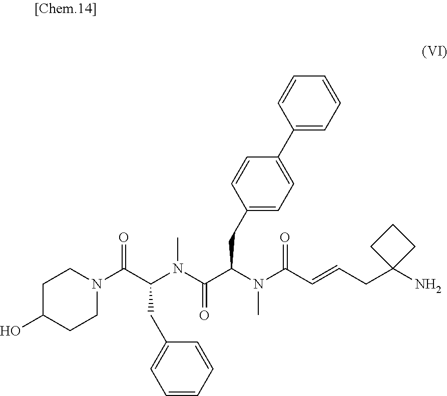 [SMILES: CN(C(=O)/C=C/CC1(N)CCC1)[C@H](Cc1ccc(-c2ccccc2)cc1)C(=O)N(C)[C@H](Cc1ccccc1)C(=O)N1CCC(O)CC1]

[9] A use of one or more selected from the group consisting of a compound of the formula (VII), a racemic-diastereomeric mixture, and an optical isomer of the said compound, and a pharmaceutically-acceptable salt and a prodrug thereof for the manufacture of a medicament for the treatment of achlorhydria in a human or an animal:

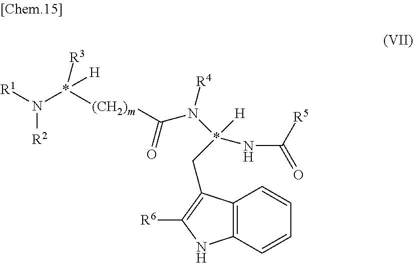

wherein * means a carbon atom which, when a chiral carbon atom, has a R or S configuration, one of R¹ and R³ is an hydrogen atom and the other is a group of formula (A)

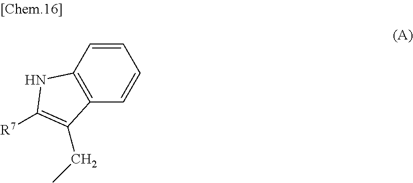

R² is a hydrogen atom, a linear or branched C₁-C₆ alkyl group, an aryl group, a heterocyclic group, a cycloalkyl group, a (CH₂)ₙ-aryl group, a (CH₂)ₙ-heterocyclic group, a (CH₂)ₙ-cycloalkyl group, a methylsulfonyl group, a phenylsulfonyl group, a C(O)R⁸ group or a group according to one of formulas (B) to (G):

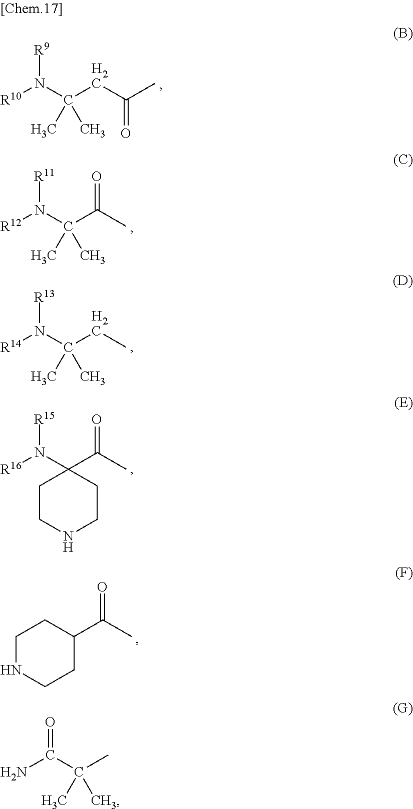

R⁴ is a hydrogen atom or a linear or branched C₁-C₄-alkyl group,

R⁵ is a hydrogen atom, a linear or branched C₁-C₄-alkyl group, a (CH₂)ₙ-aryl group, a (CH₂)ₙ-heterocyclic group, a (CH₂)-cycloalkyl group or an amino group,

R⁶ and R⁷ are independently from each other a hydrogen atom or a linear or branched C₁-C₄-alkyl group,

R⁸ is a linear or branched C₁-C₆-alkyl group, R⁹, R¹⁰, R¹¹, R¹², R¹³, R¹⁴, R¹⁵, and R¹⁶ are independently from each other a hydrogen atom or a linear or branched C₁-C₄-alkyl group, m is 0, 1 or 2 and n is 1 or 2;

[10] The use according to [9], wherein the compound is following formula (VIII):

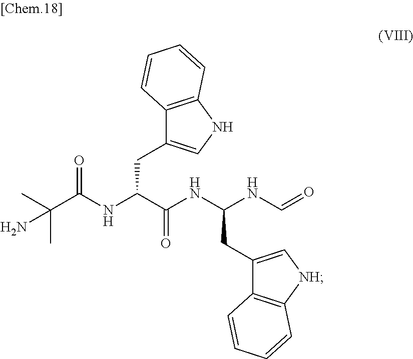 [SMILES: CC(C)(N)C(=O)N[C@H](Cc1c[nH]c2ccccc12)C(=O)N[C@H](Cc1c[nH]c2ccccc12)NC=O]

[11] A use of one or more selected from the group consisting of a compound of the formula (IX), a racemic-diastereomeric mixture, and an optical isomer of the said compound, and a pharmaceutically-acceptable salt and a prodrug thereof for the manufacture of a medicament for the treatment of achlorhydria in a human or an animal:

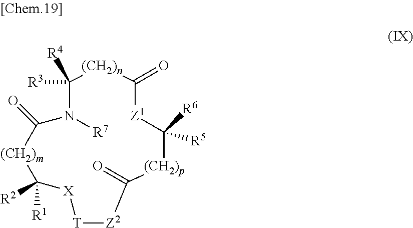

or an optical isomer, enantiomer, diastereomer, racemate or stereochemical mixture thereof, wherein:

R¹ is hydrogen or a side chain of an amino acid, or alternatively R¹ and R² together form a 4-, 5-, 6-, 7- or 8-membered ring, optionally comprising an O, S or N atom in the ring, wherein the ring is optionally substituted with R⁸ as defined below, or alternatively R¹ and R⁹ together form a 3-, 4-, 5-, 6- or 7-membered ring, optionally comprising an O, S or additional N atom in the ring, wherein the ring is optionally substituted with R⁸ as defined below;

R² is hydrogen or a side chain of an amino acid, or alternatively R¹ and R² together form a 4-, 5-, 6-, 7- or 8-membered ring, optionally comprising an O, S or N atom in the ring, wherein the ring is optionally substituted with R⁸ as defined below; or alternatively R² and R⁹ together form a 3-, 4-, 5-, 6- or 7-membered ring, optionally comprising an O, S or additional N atom in the ring, wherein the ring is optionally substituted with R⁸ as defined below;

R³ is hydrogen or a side chain of an amino acid, or alternatively R³ and R⁴ together form a 3-, 4-, 5-, 6- or 7-membered ring, optionally comprising an O or S atom in the ring, wherein the ring is optionally substituted with R⁸ as defined below, or alternatively R³ and R⁷ or R³ and R¹¹ together form a 4-, 5-, 6-, 7- or 8-membered heterocyclic ring, optionally comprising an O, S or additional N atom in the ring, wherein the ring is optionally substituted with R⁸ as defined below;
R⁴ is hydrogen or a side chain of an amino acid, or alternatively R³ and R⁴ together form a 3-, 4-, 5-, 6- or 7-membered ring, optionally comprising an O or S atom in the ring, wherein the ring is optionally substituted with R⁸ as defined below, or alternatively R⁴ and R⁷ or R⁴ and R¹¹ together form a 4-, 5-, 6-, 7- or 8-membered heterocyclic ring, optionally comprising an O, S or additional N atom in the ring, wherein the ring is optionally substituted with R⁸ as defined below;
R⁵ and R⁶ are each independently hydrogen or a side chain of an amino acid or alternatively, R⁵ and R⁶ together form a 3-, 4-, 5-, 6- or 7-membered ring, optionally comprising an O, S or N atom in the ring, wherein the ring is optionally substituted with R⁸ as defined below;
R⁷ is hydrogen, C₁-C₁₀-alkyl, substituted C₁-C₁₀-alkyl, cycloalkyl, substituted cycloalkyl, a heterocyclic group, a substituted heterocyclic group, or alternatively R³ and R⁷ or R⁴ and R⁷, together form a 4-, 5-, 6-, 7- or 8-membered heterocyclic ring optionally comprising an O, S or additional N atom in the ring, wherein the ring is optionally substituted with R⁸;
R⁸ is substituted for one or more hydrogen atoms on a 3-, 4-, 5-, 6-, 7- or 8-membered ring structure and is independently selected from the group consisting of alkyl, substituted alkyl, cycloalkyl, substituted cycloalkyl, a heterocyclic group, a substituted heterocyclic group, aryl, substituted aryl, heteroaryl, substituted heteroaryl, hydroxy, alkoxy, aryloxy, oxo, amino, halogen, formyl, acyl, carboxy, carboxyalkyl, carboxyaryl, amido, carbamoyl, guanidino, ureido, amidino, mercapto, sulfinyl, sulfonyl and sulfonamido, or, alternatively, R⁸ is a fused cycloalkyl, a substituted fused cycloalkyl, a fused heterocyclic group, a substituted fused heterocyclic group, a fused aryl, a substituted fused aryl, a fused heteroaryl or a substituted fused heteroaryl; X is O, NR⁹ or N(R¹⁰)₂⁺;
wherein R⁹ is hydrogen, C₁-C₁₀-alkyl, substituted C₁-C₁₀-alkyl, sulfonyl, sulfonamido or amidino, and R¹⁰ is hydrogen, C₁-C₁₀-alkyl, or substituted C₁-C₁₀-alkyl, or alternatively R⁹ and R¹ together form a 3-, 4-, 5-, 6- or 7-membered ring, optionally comprising an O, S or additional N atom in the ring, wherein the ring is optionally substituted with R⁸ as defined previously;

### Z¹ is O or NR¹¹;

wherein R¹¹ is hydrogen, C₁-C₁₀-alkyl, or substituted C₁-C₁₀-alkyl, or alternatively R³ and R¹¹ or R⁴ and R¹¹ together form a 4-, 5-, 6-, 7- or 8-membered heterocyclic ring, optionally comprising an O, S or additional N atom in the ring, wherein the ring is optionally substituted with R⁸ as defined above; Z² is O or NR¹²,
wherein R¹² is hydrogen, C₁-C₁₀-alkyl, or substituted C₁-C₁₀-alkyl; m, n and p are each independently 0, 1 or 2;
T is a bivalent radical of formula

—U—(CH₂)_{d}—W—Y—Z—(CH₂)ₑ—,

wherein d and e are each independently 0, 1, 2, 3, 4 or 5; Y and Z are each optionally present; U is —CR²¹R²²—, or —C(═O)— and is bonded to X of formula (IX); W, Y and Z are each independently selected from the group consisting of —O—, —NR²³—, —S—, —SO—, —SO₂—, —C(═O)—O—, —O—C(═O)—, —C(═O)—NH—, —NH—C(═O)—, —SO₂—NH—SO₂—, —CR²⁴R²⁵—, —CH═CH— with the configuration Z or E, —C≡C— and the ring structures below:

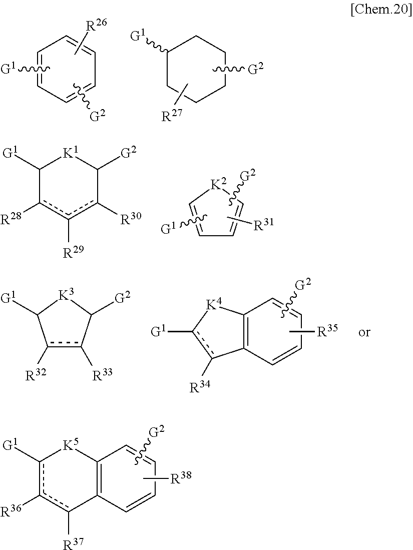

wherein G¹ and G² are each independently a bond or a bivalent radical selected from the group consisting of —O—, —NR³⁹—, —S—, —SO—, —SO₂—, —C(═O)—, —C(═O)—O—, —O—C(═O)—, —C(═O)NH—, —NH—C(═O)—, —SO₂—NH—, —NH—SO₂—, —CR⁴⁰R⁴¹—, —CH═CH— with the configuration Z or E, and —C≡C—; with G¹ being bonded closest to the group U; wherein any carbon atom in the rings not otherwise defined, is optionally replaced by N, with the proviso that the ring cannot contain more than four N atoms; K¹, K², K³, K⁴ and K⁵ are each independently O, NR⁴² or S, wherein R⁴² is as defined below;

R²¹ and R²² are each independently hydrogen, C₁-C₁₀-alkyl, or substituted C₁-C₁₀-alkyl, or alternatively R²¹ and R²² together form a 3- to 12-membered cyclic ring optionally comprising one or more heteroatoms selected from the group consisting of O, S and N, wherein the ring is optionally substituted with R⁸ as defined previously; R²³, R³⁹ and R⁴² are each independently hydrogen, alkyl, substituted alkyl, cycloalkyl, substituted cycloalkyl, a heterocyclic group, a substituted heterocyclic group, aryl, substituted aryl, heteroaryl, substituted heteroaryl, formyl, acyl, carboxyalkyl, carboxyaryl, amido, amidino, sulfonyl or sulfonamido;

R²⁴ and R²⁵ are each independently hydrogen, C₁-C₁₀-alkyl, substituted C₁-C₁₀-alkyl, R^{AA}, wherein R^{AA} is a side chain of an amino acid, or alternatively R²⁴ and R²⁵ together form a 3- to 12-membered cyclic ring optionally comprising one or more heteroatoms selected from the group consisting of O, S and N; or alternatively one of R²⁴ and R²⁵ is hydroxy, alkoxy, aryloxy, amino, mercapto, carbamoyl, amidino, ureido or guanidino while the other is hydrogen, C₁-C₁₀-alkyl or substituted C₁-C₁₀-alkyl, except when the carbon to which R²⁴ and R²⁵ are bonded is also bonded to another heteroatom;
R²⁶, R³¹ R³⁵ and R³⁸ are each optionally present and, when present, are substituted for one or more hydrogen atoms on the indicated ring and each is independently selected from the group consisting of halogen, trifluoromethyl, alkyl, substituted alkyl, cycloalkyl, substituted cycloalkyl, a heterocyclic group, a substituted heterocyclic group, aryl, substituted aryl, heteroaryl, substituted heteroaryl, hydroxy, alkoxy, aryloxy, amino, formyl, acyl, carboxy, carboxyalkyl, carboxyaryl, amido, carbamoyl, guanidino, ureido, amidino, cyano, nitro, mercapto, sulfinyl, sulfonyl and sulfonamido;
R²⁷ is optionally present and, when present, is substituted for one or more hydrogen atoms on the indicated ring and each is independently selected from the group consisting of alkyl, substituted alkyl, cycloalkyl, substituted cycloalkyl, a heterocyclic group, a substituted heterocyclic group, aryl, substituted aryl, heteroaryl, substituted heteroaryl, hydroxy, alkoxy, aryloxy, oxo, amino, formyl, acyl, carboxy, carboxyalkyl, carboxyaryl, amido, carbamoyl, guanidino, ureido, amidino, mercapto, sulfinyl, sulfonyl and sulfonamido;
R²⁸, R²⁹, R³⁰ R³², R³³, R³⁴, R³⁶ and R³⁷ are each optionally present and when no double bond is present to the carbon atom to which it is bonded in the ring, two groups are optionally present, and, when present, each is substituted for one hydrogen present in the ring, or when no double bond is present to the carbon atom to which it is bonded in the ring, is substituted for one or both of the two hydrogen atoms present on the ring and each is independently selected from the group consisting of alkyl, substituted alkyl, cycloalkyl, substituted cycloalkyl, a heterocyclic group, a substituted heterocyclic group, aryl, substituted aryl, heteroaryl, substituted heteroaryl, hydroxy, alkoxy, aryloxy, oxo, amino, formyl, acyl, carboxy, carboxyalkyl, carboxyaryl, amido, carbamoyl, guanidino, ureido, amidino, mercapto, sulfinyl, sulfonyl, sulfonamide and, only if a double bond is present, halogen; and
R⁴⁰ and R⁴¹ are each independently hydrogen, C₁-C₁₀-alkyl, substituted C₁-C₁₀-alkyl, R^{AA} as defined above, or alternatively R⁴⁰ and R⁴¹ together form a 3- to 12-membered cyclic ring optionally comprising one or more heteroatoms selected from the group consisting of O, S and N wherein the ring is optionally substituted with R⁸ as defined previously, or alternatively one of R⁴⁰ and R⁴¹ is hydroxy, alkoxy, aryloxy, amino, mercapto, carbamoyl, amidino, ureido or guanidino, while the other is hydrogen, C₁-C₁₀-alkyl or substituted C₁-C₁₀-alkyl, except when the carbon to which R⁴⁰ and R⁴¹ are bonded is also bonded to another heteroatom;
with the proviso that T is not an amino acid residue, dipeptide fragment, tripeptide fragment or higher order peptide fragment comprising standard amino acids;

[12] The use according to [11], wherein the compound is selected from following formula (Xa), (Xb), and (Xc):

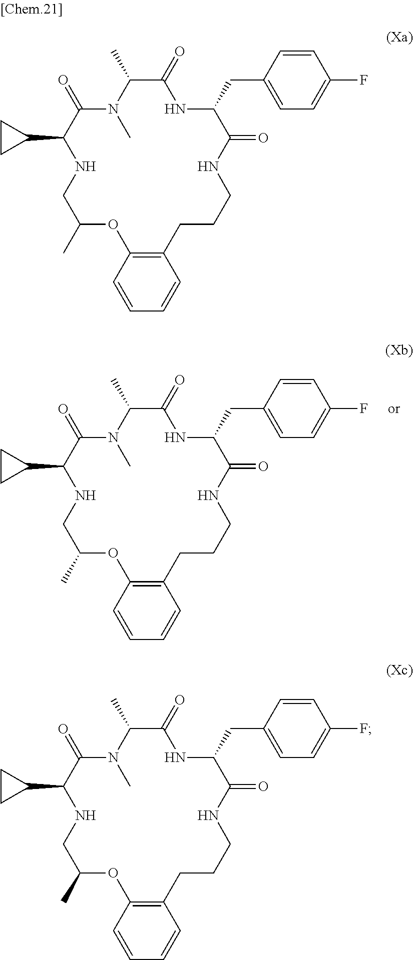 [SMILES: CC1CN[C@@H](C2CC2)C(=O)N(C)[C@H](C)C(=O)N[C@H](Cc2ccc(F)cc2)C(=O)NCCCc2ccccc2O1 ; C[C@@H]1CN[C@@H](C2CC2)C(=O)N(C)[C@H](C)C(=O)N[C@H](Cc2ccc(F)cc2)C(=O)NCCCc2ccccc2O1 ; C[C@@H]1C(=O)N[C@H](Cc2ccc(F)cc2)C(=O)NCCCc2ccccc2O[C@@H](C)CN[C@@H](C2CC2)C(=O)N1C]

[13] A use of one or more selected from the group consisting of a compound of the formula (XI), a racemic-diastereomeric mixture, and an optical isomer of the said compound, and a pharmaceutically-acceptable salt and a prodrug thereof for the manufacture of a medicament for the treatment of achlorhydria in a human or an animal:

A-B—C-D(-E)ₚ (XI)

wherein p is 0 or 1;

A is hydrogen or R¹—(CH₂)_{q}—(X)ᵣ—(CH₂)ₛ—CO—, wherein

q is 0 or an integer between 1 and 5;

r is 0 or 1;

s is 0 or an integer between 1 and 5;

R¹ is hydrogen, imidazolyl, guanidino, piperazino, morpholino, piperidino or N(R²)—R³, wherein each of R² and R³ is independently hydrogen or C₁-C₁₀-alkyl optionally substituted by one or more hydroxyl, pyridinyl or furanyl groups; and

X, when r is 1, is —NH—, —CH₂—, —CH═CH—,

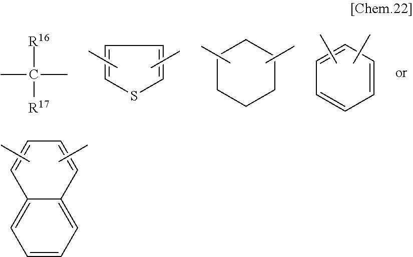

wherein each of R¹⁶ and R¹⁷ is independently hydrogen or C₁-C₁₀-alkyl;
B is (G)ₜ-(H)ᵤ wherein
t is 0 or 1;
u is 0 or 1;
G and H are amino acid residues selected from the group consisting of a natural L-amino acids or its corresponding D-isomers, and a non-natural amino acid such as 1,4-diaminobutyric acid, amino-isobutyric acid, 1,3-diaminopropionic acid, 4-aminophenylalanine, 3-pyridylalanine, 1,2,3,4-tetrahydroisoquinoline-3-carboxylic acid, 1,2,3,4-tetrahydronorharman-3-carboxylic acid, N-methylanthranilic acid, anthranilic acid, N-benzylglycine, 3-amino-3-methylbenzoic acid, 3-amino-3-methyl butanoic acid, sarcosine, nipecotic acid or iso-nipecotic acid; and wherein, when both t and u are 1, the amide bond between G and H is optionally substituted by

Y—NR¹⁸—, wherein Y is —CO— or —CH₂—, and R¹⁸ is hydrogen, C₁-C₁₀-alkyl or lower aralkyl;

C is a D-amino acid of formula —NH—CH((CH₂)_{w}—R⁴)—CO— wherein

w is 0, 1 or 2; and

R⁴ is selected from the group consisting of

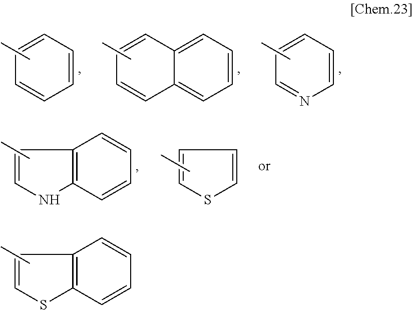

each of which is optionally substituted with halogen, C₁-C₁₀-alkyl, C₁-C₁₀-alkyloxy, C₁-C₁₀-alkylamino, amino or hydroxy;

D, when p is 1, is a D-amino acid residue of formula —NH—CH((CH₂)ₖ—R⁵)—CO— or, when p is 0, D is —NH—CH((CH₂)₁—R⁵)—CH₂—R⁶ or —NH—CH((CH₂)ₘ—R⁵)—CO—R⁶, wherein

k is 0, 1 or 2;

l is 0, 1 or 2;

m is 0, 1 or 2;

R⁵ is selected from the group consisting of

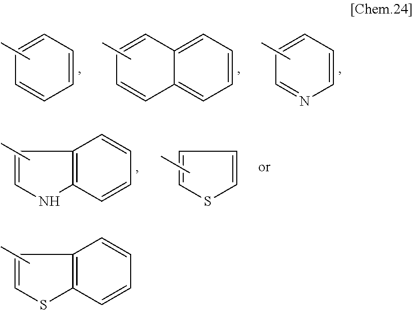

each of which is optionally substituted with halogen, alkyl, alkyloxy amino or hydroxy; and

R⁶ is piperazino, morpholino, piperidino, —OH or —N(R⁷)—R⁸, wherein each of R⁷ and R⁸ is independently hydrogen or C₁-C₁₀-alkyl;

E, when p is 1, is —NH—CH(R¹⁰)—(CH₂)ᵥ—R⁹, wherein v is 0 or an integer between 1 and 8;

R⁹ is hydrogen, imidazolyl, guanidino, piperazino, morpholino, piperidino,

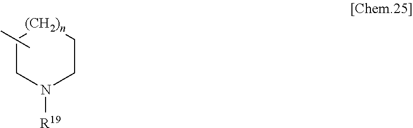

wherein n is 0, 1 or 2, and R¹⁹ is hydrogen or C₁-C₁₀-alkyl,

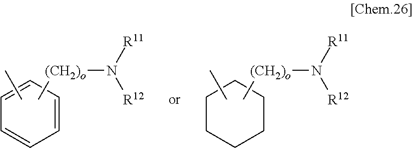

wherein o is an integer from 1 to 3,

or N(R¹¹)—R¹², wherein each of R¹¹ and R¹² is independently hydrogen or C₁-C₁₀-alkyl, or

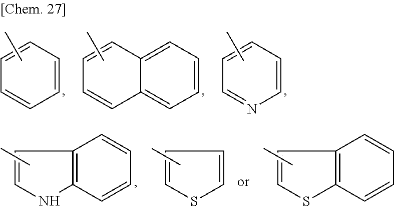

each of which is optionally substituted with halogen, alkyl, alkyloxy, amino, alkylamino, hydroxy, or the Amadori rearrangement product from an amino group and a residue formed by eliminating hydrogen from a hexapyranose or a hexapyranosyl-hexapyranose

and

R¹⁰, when p is 1, is selected from the group consisting of —H, —COON, —CH₂—R¹³, —CO—R¹³ or —CH₂—OH, wherein

R¹³ is piperazino, morpholino, piperidino, —OH or —N(R¹⁴)—R¹⁵, wherein each of R¹⁴ and
R¹⁵ is independently hydrogen or C₁-C₁₀-alkyl;
the amide bond between B and C or, when t and u are both 0, between A and C being optionally substituted by R¹⁸ or
Y—NR¹⁸—, wherein Y is —CO— or —CH₂—, and R¹⁸ is hydrogen, C₁-C₁₀-alkyl or lower aralkyl,
or, when p is 1, the amide bond between D and E being optionally substituted by Y—NR¹⁸—, wherein Y and R¹⁸ are as indicated above;
or a pharmaceutically acceptable salt thereof;

[14] The use according to [13], wherein the compound is following formula (XII):

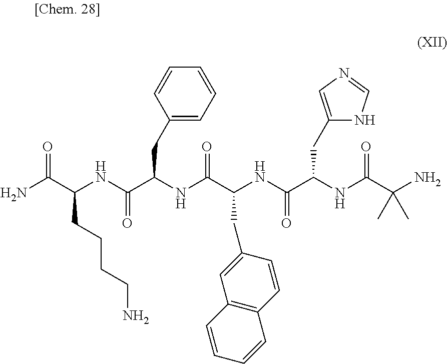 [SMILES: CC(C)(N)C(=O)N[C@@H](Cc1cnc[nH]1)C(=O)N[C@H](Cc1ccc2ccccc2c1)C(=O)N[C@H](Cc1ccccc1)C(=O)N[C@@H](CCCCN)C(N)=O]

[15] The use according to any one of [1] to [14], wherein the molecular weight of the compound is lower than 800;

[16] The use according to any one of [1] to [15], wherein the achlorhydria is age-associated achlorhydria that accompanies the aging process; chronic gastritis-associated achlorhydria; anemic achlorhydria that accompanies the anemic condition; partial gastrectomy-associated achlorhydria; calcium absorption-associated achlorhydria; vitamin D absorption-associated achlorhydria; calcitonin synthesis-associated achlorhydria; and drug-induced achlorhydria;

[17] A use of a compound or a pharmaceutically acceptable salt thereof identified in any one of [1] to [15] in combination with one or more second active agents;

[18] The use according to [17], wherein the second active agents are any of one of agents selected from:

(i) a histamine H₂ receptor antagonist, (ii) a proton pump inhibitor, (iii) an oral antacid mixture, (iv) a mucosal protective agent, (v) an anti-gastric agent, (vi) a 5-HT3 antagonist, (vii) a 5-HT4 agonist, (viii) a laxative, (ix) a GABAB agonist, (x) a GABAB antagonist, (xi) a calcium channel blocker, (xii) a dopamine antagonist, (xiii) a Tachykinin (NK) antagonist, (xiv) a Helicobacter pylori infection agent, (xv) a nitric oxide synthase inhibitor, (xvi) a vanilloid receptor 1 antagonist, (xvii) a muscarinic receptor antagonist, (xviii) a calmodulin antagonist, (xix) a potassium channel agonist, (xx) a beta-1 agonist, (xxi) a beta-2 agonist, (xxii) a beta agonist,

(xxiii) an alpha 2 agonist, (xxiv) an endothelin A antagonist, (x) an opioid μ agonist, (xxvi) an opioid p antagonist, (xxvii) a motilin agonist, (xxviii) a ghrelin agonist, (xxix) an AchE release stimulant, (xxx) a CCK-B antagonist, (xxxi) a glucagon antagonist, (xxxii) piperacillin, Ienampicillin, tetracycline, metronidazole, bithmuth citrate and bithmuth subs alicylate, (xxxiii) a Glucagon-like peptide-1 (GLP-1) antagonist, (xxxiv) a small conductance calcium-activated potassium channel 3 (SK-3) antagonist, (xxxv) a mGluR5 antagonist, (xxxvi) a 5-HT3 agonist, (xxxvii) a mGluR8 agonist, (xxxviii) a chemotherapeutic agent, (xxxix) an immunotherapeutic agent, (xL) a drug for cachexia, (xLi) a diuretic agent, and (xLii) an antidepressant;

[19] A method for the treatment of achlorhydria, which comprises administering an effective amount of a compound or a pharmaceutically acceptable salt thereof identified in any one of [1] to [15] to a human or an animal;

[20] A pharmaceutical composition comprising a compound or a pharmaceutically acceptable salt thereof identified in any one of [1] to [15] for the treatment of achlorhydria;

[21] A kit for the treatment of achlorhydria, comprising a compound or a pharmaceutically acceptable salt thereof identified in any one of [1] to [15];

[22] The kit according to [21], which comprises a compound or a pharmaceutically acceptable salt thereof identified in any one of [1] to [15], at least one second active agent, and a container; and

[23] A commercial package comprising a pharmaceutical composition containing a compound or a pharmaceutically acceptable salt thereof identified in any one of [1] to [15] and a written matter associated with said pharmaceutical composition, the written matter stating that said pharmaceutical composition can or should be used for treating achlorhydria.

In the above structural formulae and throughout the instant application, heteroaryl may be abbreviated as hetaryl, and the following terms have the indicated meanings unless expressly stated otherwise:

The alkyl groups are intended to include those alkyl groups of the designated length in either a straight or branched configuration which may optionally contain double or triple bonds. Examples of such alkyl groups are methyl, ethyl, propyl, isopropyl, butyl, sec-butyl, tertiary butyl, pentyl, isopentyl, hexyl, isohexyl, allyl, ethynyl, propenyl, butadienyl, hexenyl and the like.

When the definition C₀-alkyl occurs in the definition, it means a single bond.

The alkoxy groups specified above are intended to include those alkoxy groups of the designated length in either a straight or branched configuration which may optionally contain double or triple bonds. Examples of such alkoxy groups are methoxy, ethoxy, propoxy, isopropoxy, butoxy, isobutoxy, tertiary butoxy, pentoxy, isopentoxy, hexoxy, isohexoxy, allyloxy, 2-propynyloxy, isobutenyloxy, hexenyloxy and the like.

The term “halogen” or “halo” is intended to include the halogen atoms fluorine, chlorine, bromine and iodine.

The term “alogenated alkyl” is intended to include an alkyl group as defined hereinabove substituted by one or more halogen atoms as defined hereinabove.

The term “halogenated cycloalkyl” is intended to include a cycloalkyl group substituted by one or more halogen atoms as defined hereinabove.

The term “aryl” is intended to include phenyl and naphthyl and a substituent formed by eliminating hydrogen from aromatic 5- and 6-membered ring with 1 to 4 heteroatoms or fused 5- or 6-membered bicyclic ring with 1 to 4 heteroatoms of nitrogen, sulfur and/or oxygen. Examples of such heterocyclic aromatic rings are pyridine, thiophene (also known as thienyl), furan, benzothiophene, tetrazole, indole, N-methylindole, dihydroindole, indazole, N-formylindole, benzimidazole, thiazole, pyrimidine, and thiadiazole.

The term “achlorhydria”, as used herein, includes various type of achlorhydria, not limited to, age-associated achlorhydria that accompanies the aging process; chronic gastritis-associated achlorhydria; anemic achlorhydria that accompanies the anemic condition; partial gastrectomy-associated achlorhydria; calcium absorption-associated achlorhydria; vitamin D absorption-associated achlorhydria; calcitonin synthesis-associated achlorhydria; and drug (such as PPI)-induced achlorhydria.

The term “treating” or “treatment”, as used herein, refers to reversing, alleviating, inhibiting the progress of, or preventing the disorder or condition to which such term applies, or one or more symptoms of such disorder or condition. It includes not only treatment of achlorhydria but also alleviation of symptoms, improvement of QOL, and prophylaxis. Therefore it includes “therapeutic agent” and “prophylactic agent”.

Those skilled in the art will recognize that certain combinations of heteroatom-containing substituents listed in this invention define compounds which will be less stable under physiological conditions (e.g., those containing acetal or aminal linkages). Accordingly, such compounds are less preferred.

### BRIEF DESCRIPTION OF DRAWINGS

FIG. 1 shows the effect on a vehicle against intra-gastric pH.

FIG. 2 shows the effect on a ghrelin receptor agonist (compound A) against intra-gastric pH.

### DESCRIPTION OF EMBODIMENTS

The inventors started to find or prepare compounds which increase gastric-acid secretion, which can alleviate achlorhydria. After an exhaustive and careful study, the inventors of the present invention have managed to find out that compounds which have agonistic activities against ghrelin receptor have dramatically decreased the intra-gastric pH value and maintained the low value for a long period. Thus compounds of the present invention enhanced gastric acid secretion, which provides the much-anticipated drugs for the treatment of achlorhydria.

It has been established that a ghrelin receptor agonist has a lot of pharmacological use (White H. K., Petrie C. D., Landschulz W., et Al., J. Clin. Endocrinol. Metab., 94:1198-1206, 2009; Garcia J. M. and Polvino W. J., The Oncologist, 12:594-600, 2007; Nagaya N., Kojima M., Uematsu M., Yamagishi M., Hosoda H., Oya H., Hayashi Y., and Kangawa K., Am. J. Physiol. Regulatory. Integrative Comp. Physiol., 280: R148-R1487, 2001; De Smet B., Mitselos A., and Depoortere I., Pharmacology & Therapeutics, 123: 207-223, 2009). However, under the current status of research in this area, the fact that the ghrelin receptor agonist enhances gastric acid secretion has never been found so far. Therefore, the use of ghrelin receptor agonists for achlorhydria can not be foreseen for those skilled in the art.

One of more compounds of the present invention may be usefully combined with another pharmacologically active compound, or with two or more other pharmacologically active compounds (second active agents).

For example, a ghrelin receptor agonist of the present invention, or a pharmaceutically acceptable salt thereof, as defined above, may be administered simultaneously, sequentially or separately in combination with one or more agents selected from:

(i) histamine H₂ receptor antagonists, e.g. ranitidine, lafutidine, nizatidine, cimetidine, famotidine and roxatidine;

(ii) proton pump inhibitors, e.g. omeprazole, esomeprazole, pantoprazole, rabeprazole, tenatoprazole, ilaprazole and lansoprazole;

(iii) oral antacid mixtures, e.g. Maalox(registered trade mark), Aludrox(registered trade mark) and Gaviscon(registered trade mark);

(iv) mucosal protective agents, e.g. polaprezinc, ecabet sodium, rebamipide, teprenone, cetraxate, sucralfate, chlorophylline-copper and plaunotol;

(v) anti-gastric agents, e.g. Anti-gastrin vaccine, itriglumide and Z-360;

(vi) 5-HT₃ antagonists, e.g. dolasetron, palonosetron, alosetron, azasetron, ramosetron, mirtazapine, granisetron, tropisetron, E-3620, ondansetron and indisetron;

(vii) 5-HT₄ agonists, e.g. tegaserod, mosapride, cinitapride and oxtriptan;

(viii) laxatives, e.g. Trifyba(registered trade mark), Fybogel(registered trade mark), Konsyl(registered trade mark), Isogel(registered trade mark), Regulan(registered trade mark), Celevac(registered trade mark) and Normacol(registered trade mark);

(ix) GABAB agonists, e.g. baclofen and AZD-3355;

(x) GABAB antagonists, e.g. GAS-360 and SGS-742;

(xi) calcium channel blockers, e.g. aranidipine, lacidipine, felodipine, azelnidipine, cilnidipine, lomerizine, diltiazem, gallopamil, efonidipine, nisoldipine, amlodipine, lercanidipine, bevantolol, nicardipine, isradipine, benidipine, verapamil, nitrendipine, barnidipine, propafenone, manidipine, bepridil, nifedipine, nilvadipine, nimodipine and fasudil;

(xii) dopamine antagonists, e.g. metoclopramide, domperidone and levosulpiride;

(xiii) Tachykinin (NK) antagonists, particularly NK-3, NK-2 and NK-1 antagonists, e.g. nepadutant, saredutant, talnetant, (αR,9R)-7-[3,5-bis(trifluoromethyl)benzyl]-8,9,10,11-tetrahydro-9-methyl-5-(4-methylphenyl)-7H-[1,4]diazocino[2,1-g][1,7]naphthyridine-6,13-dione (TAK-637), 5-[[(2R,3S)-2-[(1R)-1-[3,5-bis(trifluoromethyl)phenyl]ethoxy-3-(4-fluorophenyl)-4-morpholinyl]methyl]-1,2-dihydro-3H-1,2,4-triazol-3-one (MK-869), lanepitant, dapitant and 3-[[2-methoxy-5-(trifluoromethoxy)phenyl]methylamino]-2-phenyl-piperidine (2S,3S);

(xiv) Helicobacter pylori infection agents, e.g. clarithromycin, roxithromycin, rokitamycin, flurithromycin, telithromycin, amoxicillin, ampicillin, temocillin, bacampicillin, aspoxicillin, sultamicillin, piperacillin, lenampicillin, tetracycline, metronidazole, bithmuth citrate and bithmuth subsalicylate;

(xv) nitric oxide synthase inhibitors, e.g. GW-274150, tilarginine, P54, guanidinoethyldisulfide and nitroflurbiprofen;

(xvi) vanilloid receptor 1 antagonists, e.g. AMG-517 and GW-705498; (xvii) muscarinic receptor antagonists, e.g. trospium, solifenacin, tolterodine, tiotropium, cimetropium, oxitropium, ipratropium, tiquizium, darifenacin and imidafenacin;

(xviii) calmodulin antagonists, e.g. squalamine and DY-9760;

(xix) potassium channel agonists, e.g. pinacidil, tilisolol, nicorandil, NS-8 and retigabine;

(xx) beta-1 agonists, e.g. dobutamine, denopamine, xamoterol, denopamine, docarpamine and xamoterol;

(xxi) beta-2 agonists, e.g. salbutamol; terbutaline, arformoterol, meluadrine, mabuterol, ritodrine, fenoterol, clenbuterol, formoterol, procaterol, tulobuterol, pirbuterol, bambuterol, tulobuterol, dopexamine and levosalbutamol;

(xxii) beta agonists, e.g. isoproterenol and terbutaline;

(xxiii) alpha 2 agonists, e.g. clonidine, medetomidine, lofexidine, moxonidine, tizanidine, guanfacine, guanabenz, talipexole and dexmedetomidine;

(xxiv) endothelin A antagonists, e.g. bonsetan, atrasentan, ambrisentan, clazosentan, sitaxsentan, fandosentan and darusentan;

(xxv) opioid p agonists, e.g. morphine, fentanyl and loperamide;

(xxvi) opioid p antagonists, e.g. naloxone, buprenorphine and alvimopan;

(xxvii) motilin agonists, e.g. erythromycin, mitemcinal, SLV-305 and atilmotin;

(xxviii) ghrelin agonists, e.g. capromorelin and TZP-101;

(xxix) AchE release stimulants, e.g. Z-338 and KW-5092;

(xxx) CCK-B antagonists, e.g. itriglumide, YF-476 and S-0509;

(xxxi) glucagon antagonists, e.g. NN-2501 and A-770077;

(xxxii) piperacillin, Ienampicillin, tetracycline, metronidazole, bithmuth citrate and bithmuth subsalicylate;

(xxxiii) Glucagon-like peptide-1 (GLP-1) antagonists, e.g. PNU-126814;

(xxxiv) small conductance calcium-activated potassium channel 3 (SK-3) antagonists, e.g. apamin, dequalinium, atracurium, pancuronium and tubocurarine;

(xxxv) mGluR5 antagonists, e.g. ADX-10059 and AFQ-056;

(xxxvi) 5-HT3 agonists, e.g. pumosetrag(DDP733);

(xxxvii) mGluR8 agonists, e.g. (S)-3,4-DCPG and mGluR8-A;

(xxxviii) chemotherapeutic agents, e.g. alkylating agents (e.g. cyclophosphamide, ifos famide), antimetabolites (e.g. methotrexate, 5-fluorouracil), antitumor antibiotics (e.g. mitomycin, adriamycin), antitumor plant alkaloids (e.g. vincristine, vindesine, Taxol), cisplatin, carboplatin, and etoposide.

Particularly preferred are Flutron and Neo-Flutron, which are 5-fluorouracil derivatives;

(xxxix) immunotherapeutic agents, e.g. fungal or bacterial cell wall components (e.g. muramyl dipeptide derivatives, picibanil), immunostimulant polysaccharides (e.g. lentinan, schizophyllan, Krestin), recombinant cytokines (e.g. interferons, interleukins (IL)), and colony stimulating factors (e.g. granulocyte colony stimulating factor, erythropoietin), particularly preferred are IL-1, IL-2, and IL-12;

(xL) drugs for cachexia, e.g. cyclooxygenase inhibitors (e.g. indomethacin) [Cancer Research, 49, 5935-5939, 1989], progesterone derivatives (e.g. megestrol acetate) [Journal of Clinical Oncology, 12. 213-225, 1994], glucocorticoids (e.g. dexamethasone), metoclopramides, tetrahydrocannabinols (the same literature as above), lipid metabolism improving agents (e.g. eicosapentanoic acid) [British Journal of Cancer, 68, 314-318, 1993], growth hormone, IGF-1, and antibodies to the cachexia-inducing factors TNF-α, LIF, IL-6, and oncostatin M;

(xLi) diuretic agents, e.g. xanthine derivative preparations (e.g. theobromine and sodium salicylate, theobromine and calcium salicylate), thiazide preparations (e.g. ethiazide, cyclopenthiazide, trichlormethiazide, hydrochlorothiazide, hydroflumethiazide, benzylhydrochlorothiazide, penflutizide, polythiazide, methyclothiazide), antialdosterone preparations (e.g. spironolactone, triamterene), carbonate dehydratase inhibitors (e.g. acetazolamide), chlorbenzene sulfonamide preparations (e.g. chlorthalidone, mefruside, indapamide), azosemide, isosorbide, ethacrynic acid, piretanide, bumetanide, and furosemide; and

(xLii) antidepressants, e.g. citalopram hydrobromide, escitalopram oxalate, fluvoxamine maleate, paroxetine hydrochloride, paroxetine mesylate, sertraline hydrochloride, and Mirtazapine.

In terms of pharmaceutically acceptable acid addition salts, suitable acid addition salts are formed from acids which form non-toxic salts. Examples include acetate, aspartate, benzoate, besylate, bicarbonate/carbonate, bisulphate/sulphate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, saccharate, stearate, succinate, tartrate, tosylate and trifluoroacetate salts.

Suitable base salts are formed from bases which form non-toxic salts. Examples include the aluminum, arginine, benzathine, calcium, choline, diethylamine, diolamine, glycine, lysine, magnesium, meglumine, olamine, potassium, sodium, tromethamine and zinc salts.

For a review on suitable salts, see “Handbook of Pharmaceutical Salts: Properties, Selection, and Use” by Stahl and Wermuth (Wiley-VCH, Weinheim, Germany, 2002).

A pharmaceutically acceptable salt of a compound of the present invention may be readily prepared by mixing together solutions of the compound and the desired acid or base, as appropriate. The salt may precipitate from solution and be collected by filtration or may be recovered by evaporation of the solvent. The degree of ionization in the salt may vary from completely ionized to almost non-ionized.

Pharmaceutically acceptable salts of the compounds of the present invention include both unsolvated and solvated forms. The term “solvate” is used herein to describe a molecular complex comprising a compound of the invention and one or more pharmaceutically acceptable solvent molecules, for example, ethanol.

Included within the scope of the invention are complexes such as clathrates, and drug-host inclusion complexes wherein, in contrast to the aforementioned solvates, the drug and host are present in stoichiometric or non-stoichiometric amounts. Also included are complexes of the drug containing two or more organic and/or inorganic components which may be in stoichiometric or non-stoichiometric amounts. The resulting complexes may be ionized, partially ionized, or non-ionized. For a review of such complexes, see J Pharm Sd. 64 (8), 1269-1288 by Haleblian (August 1975).

All references to a compound of the present invention include references to salts and complexes thereof and to solvates and complexes of salts thereof.

A compound of the present invention includes polymorphs, prodrugs, and isomers thereof (including optical, geometric and tautomeric isomers) and isotopically-labeled compounds of the present invention as herein defined.

As mentioned above, this invention includes all polymorphs as hereinbefore defined.

Also within the scope of the invention are so-called “prodrugs” of the compounds of formula (I). Thus certain derivatives of compounds of formula (I) which may have little or no pharmacological activity themselves can, when administered into or onto the body, be converted into compounds of formula (I) having the desired activity, for example, by hydrolytic cleavage. Such derivatives are referred to as “prodrugs”. Further information on the use of prodrugs may be found in Pro-drugs as Novel Delivery Systems, Vol. 14, ACS Symposium Series (T Higuchi and W Stella) and Bioreversible Carriers in Drug Design, Pergamon Press, 1987 (ed. E B Roche, American Pharmaceutical Association).

Prodrugs in accordance with the invention can, for example, be produced by replacing appropriate functionalities present in the compounds of formula (I) with certain moieties known to those skilled in the art as ‘pro-moieties’ as described, for example, in Design of Prodrugs by H Bundgaard (Elsevier, 1985).

Some examples of prodrugs in accordance with the invention include:

(i) where the compound of this invention contains a carboxylic acid functionality (—COOH), an ester thereof, for example, replacement of the hydrogen of —COOH with (C₁-C₆)alkanoyloxymethyl;

(i) where the compound of this invention contains an alcohol functionality (—OH), an ether thereof, for example, replacement of the hydrogen of —OH with (C₁-C₆)alkanoyloxymethyl; and

(iii) where the compound of formula (I) contains a primary or secondary amino functionality (—NH₂ or —NHR where R is not H but a substituent), an amide thereof, for example, replacement of one or both hydrogens of —NH₂ or NHR with (C₁-C₁₀)alkanoyl.

Further examples of replacement groups in accordance with the foregoing examples are well known in the art and examples of other prodrug types may be found in the aforementioned references, but are not limited to these.

Certain compounds of the present invention may themselves act as prodrugs of other compounds of this invention.

Compounds of this invention containing one or more asymmetric carbon atoms can exist as two or more stereoisomers. Where a compound of this invention contains an alkenyl or alkenylene group, geometric cis/trans (or Z/E) isomers are possible. Where the compound contains, for example, a keto or oxime group, an aromatic moiety or a heteroaromatic ring including nitrogen of more than two, tautomeric isomerism (‘tautomerism’) can occur. It follows that a single compound may exhibit more than one type of isomerism.

Included within the scope of the present invention are all stereoisomers, geometric isomers and tautomeric forms of the compounds of this invention, including compounds exhibiting more than one type of isomerism, and mixtures of one or more thereof. Also included are acid addition or base salts wherein the counter ion is optically active, for example, D-lactic acid or L-lysine, or racemic, for example, DL-tartaric acid or DL-arginine.

Cis/trans isomers may be separated by conventional techniques well known to those skilled in the art, for example, chromatography and fractional crystallization.

Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC).

Alternatively, the racemate (or a racemic precursor) may be reacted with a suitable optically active compound, for example, an alcohol, or, in the case where the compound of formula (I) contains an acidic or basic moiety, an acid or base such as tartaric acid or 1-phenylethylamine. The resulting diastereomeric mixture may be separated by chromatography and/or fractional crystallization and one or both of the diastereoisomers converted to the corresponding pure enantiomer(s) by means well known to those skilled in the art.

Chiral compounds of the present invention (and chiral precursors thereof) may be obtained in enantiomerically-enriched form using chromatography, typically HPLC, on an asymmetric resin with a mobile phase consisting of a hydrocarbon, typically heptane or hexane, containing from 0 to 50% isopropanol, typically from 2 to 20%, and from 0 to 5% of an alkylamine, typically 0.1% diethylamine. Concentration of the eluate affords the enriched mixture.

Stereoisomeric conglomerates may be separated by conventional techniques known to those skilled in the art—see, for example, “Stereochemistry of Organic Compounds” by E L Eliel (Wiley, New York, 1994).

The present invention includes all pharmaceutically acceptable isotopically-labelled compounds of this invention wherein one or more atoms are replaced by atoms having the same atomic number, but an atomic mass or mass number different from the atomic mass or mass number usually found in nature.

Examples of isotopes suitable for inclusion in the compounds of the invention include isotopes of hydrogen, such as ²H and ³H, carbon, such as ¹¹C, ¹³C and ¹⁴C, chlorine, such as ³⁶CI, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S.

Certain isotopically-labelled compounds of formula (I), for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ⁵¹O and ¹³N, can be useful in Positron Emission Topography (PET) studies for examining substrate receptor occupancy.

Isotopically-labeled compounds of the present invention can generally be prepared by conventional techniques known to those skilled in the art or by processes analogous to those described in the accompanying Examples and Preparations using an appropriate isotopically-labeled reagents in place of the non-labeled reagent previously employed.

Pharmaceutically acceptable solvates in accordance with the invention include those wherein the solvent of crystallization may be isotopically substituted, e.g. D₂O, d₆-acetone, d₆-DMSO.

Compounds of the invention intended for pharmaceutical use may be administered as crystalline or amorphous products. They may be obtained, for example, as solid plugs, powders, or films by methods such as precipitation, crystallization, freeze drying, or spray drying, or evaporative drying. Microwave or radio frequency drying may be used for this purpose.

They may be administered alone or in combination with one or more other compounds of the invention or in combination with one or more other drugs (or as any combination thereof). Generally, they will be administered as a formulation in association with one or more pharmaceutically acceptable excipients. The term “excipient” is used herein to describe any ingredient other than the compound(s) of the invention. The choice of excipient will to a large extent depend on factors such as the particular mode of administration, the effect of the excipient on solubility and stability, and the nature of the dosage form.

Therefore the present invention provides the combination comprising a compound of the present invention, its solvate or prodrug, and a compound (or a compound group as a second active agent) selected from one or more pharmaceutically active drugs. In addition, the present invention provides a pharmaceutical composition comprising such combination together with a pharmaceutically acceptable additive, diluent or carrier, especially for the treatment of a variety of diseases caused by abnormal gastrointestinal motility. Further, the present invention provides a kit comprising a first pharmaceutical composition containing a compound of the present invention or a pharmaceutically acceptable salt thereof; a second active agent; and a container.

A kit comprising a compound of the present invention, or a pharmaceutically acceptable salt thereof for the treatment of a variety of diseases caused by abnormal gastrointestinal motility is one of the present inventions. A commercial package comprising the pharmaceutical composition containing the compound of the present invention, or a pharmaceutically acceptable salt thereof and a writtenmatter associated therewith, wherein the written matter states that the compound can or should be used for treating a variety of diseases caused by abnormal gastrointestinal motility.

The term “treating”, as used herein, refers to reversing, alleviating, inhibiting the progress of, or preventing the disorder or condition to which such term applies, or one or more symptoms of such disorder or condition. The term “treatment” as used herein includes not only treating diseases caused by abnormal gastric acid secretion but also broadly includes relieving symptoms, improving QOL and the concept of so-called prevention.

Other features and advantages of the invention will be apparent from the following detailed description and from the claims. While the invention is described in connection with specific embodiments, other changes and modifications that may be practiced are also part of this invention and are also within the scope of the appendant claims, including departures from the present disclosure that come within known or customary practice within the art. This application is intended to cover any equivalents, variations, uses, or adaptations of the invention that follow, in general, the principles of the invention.

A compound of the present invention is administered in a dose sufficient to enhance a variety of diseases caused by abnormal gastrointestinal motility. Such therapeutically effective amounts will be determined using routine optimization techniques that are dependent on the particular condition to be treated, the condition of the patient, the route of administration, the formulation, the judgment of the practitioner, and other factors evident to those skilled in the art in light of this disclosure.

A compound of the present invention can be incorporated into a therapeutic composition. Such a pharmaceutical agent are combined with a pharmaceutically acceptable delivery vehicle or carrier.

A pharmaceutically acceptable delivery vehicle includes solvents, dispersion media, coatings, antibacterial and antifungal agents, and isotonic and absorption delaying agents that are compatible with pharmaceutical administration. The vehicle may also include other active or inert components.

Therapeutic efficacy of a compound of the present invention can be determined in light of this disclosure by standard therapeutic procedures in in vitro assay such as cell cultures or experimental animals, e.g., for determining the ED50 (the dose therapeutically effective in 50% of the population).

The data obtained from the in vitro assays and animal studies can be used in formulating a range of dosage for use in humans. The dosage may vary depending upon the formulation and the route of administration. For any compounds used in the method of the invention, the therapeutically effective dose can be estimated initially from in vitro assays. A dose may be formulated in animal models to achieve a circulating plasma concentration range that includes the IC₅₀ as determined in in vitro assay. Such information can be used to more accurately determine useful doses in humans. Levels in plasma may be measured, for example, by high performance liquid chromatography and mass spectrometer.

The skilled artisan will appreciate that certain factors may influence the dosage and timing required to effectively treat a mammal including, but not limited to, the severity of the disease or disorder, previous treatments, the general health and/or age of the mammal, and other diseases present. Moreover, treatment of a mammal with a therapeutically effective amount of a compound of the present invention can include a single treatment, an intermittent treatment, or a series of treatments, but not limited to these.

Particularly the precise amount of the compounds administered to a human patient will be the responsibility of the attendant physician. However, the dose employed will depend upon a number of factors including the age and sex of the patient, the precise condition being treated and its severity, and the route of administration.

The compounds of the present invention are conveniently administered in the form of pharmaceutical compositions. Such compositions may conveniently be presented for use in conventional manner in admixture with one or more physiologically acceptable carriers or excipients. Compositions comprising a compound of the present invention can be also one of the inventions.

While it is possible for the compounds of the present invention to be administered as the raw chemical, it is preferable to present it as a pharmaceutical formulation. The formulations comprise the compounds together with one or more acceptable carriers or diluents therefor and optionally other therapeutic ingredients. The carrier(s) must be acceptable in the sense of being compatible with the other ingredients of the formulation and not deleterious to the recipient thereof.

A therapeutic composition is formulated to be compatible with its intended route of administration. Non-limiting examples of routes of administration include parenteral, e.g., intravenous, intradermal, subcutaneous, oral (e.g., by ingestion or inhalation), transdermal (topical), transmucosal, and rectal administration. Solutions or suspensions can be made as described in Remington's Pharmaceutical Sciences, (181 1 ed., Gennaro, ed., Mack Publishing Co., Easton, Pa., (1990)).

The most suitable route may depend upon for example the condition and disorder of the recipient. The formulations may conveniently be presented in unit dosage form and may be prepared by any of the methods well known in the art of pharmacy. All methods include the step of bringing into association the compounds (“active ingredient”) with the carrier which constitutes one or more accessory ingredients. In general the formulations are prepared by uniformly and intimately bringing into association the active ingredient with liquid carriers or finely divided solid carriers or both and then, if necessary, shaping the product into the desired formulation.

Formulations suitable for oral administration may be presented as discrete units such as capsules, cachets or tablets (e.g. chewable tablets in particular for paediatric administration) each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

A tablet may be made by compression or moulding, optionally with one or more accessory ingredients. Compressed tablets may be prepared by compressing in a suitable machine the active ingredient in a free-flowing form such as a powder or granules, optionally mixed with a binder, lubricant, inert diluent, lubricating, surface active or dispersing agent. Moulded tablets may be made by moulding in a suitable machine a mixture of the powdered compound moistened with an inert liquid diluent. The tablets may optionally be coated or scored and may be formulated so as to provide slow or controlled release of the active ingredient therein.

Formulations for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilised) condition requiring only the addition of a sterile liquid carrier, for example, water-for-injection, immediately prior to use. Extemporaneous injection solutions and suspensions may be prepared from sterile powders, granules and tablets of the kind previously described.

Formulations for rectal administration may be presented as a suppository with the usual carriers such as cocoa butter, hard fat or polyethylene glycol.

Formulations for topical administration in the mouth, for example buccally or sublingually, include lozenges comprising the active ingredient in a flavoured basis such as sucrose and acacia or tragacanth, and pastilles comprising the active ingredient in a basis such as gelatin and glycerin or sucrose and acacia.

The compounds of the present invention or pharmaceutically acceptable salts thereof may also be formulated as depot preparations. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

In addition to the ingredients particularly mentioned above, the formulations may include other agents conventional in the art having regard to the type of formulation in question, for example those suitable for oral administration may include flavouring agents.

The present invention also relates to combining separate pharmaceutical compositions in kit form. The kit comprises two separate pharmaceutical compositions: a compound of the present invention, a prodrug thereof or a pharmaceutically acceptable salt of said compound or said prodrug; and a second therapeutic agent as described herein. The kit comprises a container for containing the separate compositions such as a divided bottle or a divided foil packet, however, the separate compositions may also be contained within a single, undivided container. The kit form is particularly advantageous when the separate components are preferably administered in different dosage forms (e.g., oral and parenteral), are administered at different dosage intervals, or when titration of the individual components of the combination is desired by the prescribing physician.

An example of such a kit is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process, recesses are formed in the plastic foil. The recesses have the size and shape of the tablets or capsules to be packed. Next, the tablets or capsules are placed in the recesses and the sheet of relatively stiff material is sealed against the plastic foil at the face of the foil which is opposite from the direction in which the recesses were formed. As a result, the tablets or capsules are sealed in the recesses between the plastic foil and the sheet. Preferably, the strength of the sheet is such that the tablets or capsules can be removed from the blister pack by manually applying pressure on the recesses whereby an opening is formed in the sheet at the place of the recess. The tablet or capsule can then be removed via said opening.

Exemplary Methods of Combination Therapy

In certain embodiments, the methods provided herein comprise administering a compound of the present invention in combination with one or more second active agents, and/or in combination with radiation therapy or surgery. The administration of a compound of the present invention and the second active agents to a patient can occur simultaneously or sequentially by the same or different routes of administration. The suitability of a particular route of administration employed for a particular active agent will depend on the active agent itself (e.g., whether it can be administered orally without decomposing prior to entering the blood stream) and the disease being treated. Recommended routes of administration for the second active agents are known to those of ordinary skill in the art. See, e.g., Physicians' Desk Reference.

In one embodiment, a compound of the present invention or the second active agent is administered intravenously or subcutaneously and once or twice daily in an amount of from about 0.1 to about 3,000 mg, preferably from about 1 to about 1,000 mg, more preferably from about 5 to about 500 mg or most preferably from about 10 to about 375 mg or further most preferably from about 50 to about 200 mg.

In another embodiment, provided herein are methods of treating, preventing and/or managing a variety of diseases caused by abnormal gastrointestinal motility, such as achlorhydria which comprise administering a compound of the present invention in conjunction with (e.g., before, during or after) conventional therapy including, but not limited to, other non-drug based therapy presently used. Without being limited by theory, it is believed that a compound of the present invention may provide additive or synergistic effects when given concurrently with conventional therapy.

In certain embodiments, the second active agent is co-administered with a compound of the present invention or administered with 1 to 50 hours delay. In certain embodiments, a compound of the present invention is administered first followed by administration with the second active agent with 1 to 50 hours delay. In other embodiments, the second active agent is administered first followed by administration of a compound of the present invention with 1 to 50 hours delay. In some embodiment, the delay is 24 hours.

In one embodiment, a compound of the present invention can be administered in an amount of from about 0.1 to about 3,000 mg/day alone or in combination with a second active agent disclosed herein, prior to, during, or after the use of conventional therapy.

In another embodiment, the methods provided herein comprise: a) administering to a patient in need thereof, a dose of about 0.1 to about 3,000 mg/day of a compound of the present invention and b) administering a therapeutically effective amount of a second active agent such as a supportive care agent.

The administration mode of the compound of the present invention and the concomitant drug is not particularly limited, and the compound of the present invention and the concomitant drug only need to be combined on administration. Examples of such administration mode include the following:

(1) administration of a single preparation obtained by simultaneously processing the compound of the present invention and the concomitant drug, (2) simultaneous administration of two kinds of preparations of the compound of the present invention and the concomitant drug, which have been separately produced, by the same administration route, (3) administration of two kinds of preparations of the compound of the present invention and the concomitant drug, which have been separately produced, by the same administration route in a staggered manner, (4) simultaneous administration of two kinds of preparations of the compound of the present invention and the concomitant drug, which have been separately produced, by different administration routes, (5) administration of two kinds of preparations of the compound of the present invention and the concomitant drug, which have been separately produced, by different administration routes in a staggered manner (e.g., administration in the order of the compound of the present invention and the concomitant drug, or in the reverse order) and the like. In the following, these administration modes are collectively abbreviated as the concomitant drug of the present invention.

When the compounds of the present invention are used in combination with one or more other therapeutic agents (second active agents), the compounds may be administered either sequentially or simultaneously by any convenient route.

The combinations referred to above may conveniently be presented for use in the form of a pharmaceutical formulation and thus pharmaceutical formulations comprising a combination as defined above together with a pharmaceutically acceptable carrier or excipient comprise a further aspect of the invention. The individual components of such combinations may be administered either sequentially or simultaneously in separate or combined pharmaceutical formulations.

When a compound of the present invention is used in combination with a second therapeutic agent active against the same disease, the dose of each compound may differ from that when the compound is used alone. Appropriate doses will be readily appreciated by those skilled in the art.

Preferred unit dosage formulations are those containing an effective daily dose, as herein above recited, or an appropriate fraction thereof, of the active ingredient. For example, a daily dose of a compound of the present invention may be from 0.1 mg to 3,000 mg, more preferably about 1 mg to 1,000 mg. As mentioned above, a dose may depend on the condition of individual patients, and is not limited to these.

Suitable subject to be administered a compound of the present invention activity or a pharmaceutical composition containing the compound is a mammal, including humans. Among them, a mammal with a variety of diseases caused by abnormal gastric acid is preferable. A mammal with high gastric pH caused by surpressed gastric acid secretion is more preferable.

### EXAMPLE

The compounds described in the present invention are known to the public and can be synthesized by the known method. The following patent applications, for example, WO97/24369, WO1998/008492, WO1999/058501, WO2000/01726, WO2000/74702, and WO2008/100448 are referred.

Compound A:
2-amino-N-[2-(3a-(R)-benzyl-2-methyl-3-oxo-2,3,3a,4,6,7-hexahydropyrazolo[4,3-c]pyridin-5-yl)-1-(R)-benzyloxymethyl-2-oxo-ethyl]isobutyramide.

Compound B:
2-amino-N-[1-(R)-(2,4-difluoro-benzyloxymethyl)-2-oxo-2-(3-oxo-3a-(R)-pyridin-2-yl methyl-2-(2,2,2-trifluoro-ethyl)-2,3,3a,4,6,7-hexahydropyrazolo[4,3-c]pyridin-5-yl)-ethyl]-2-methyl-propionamide.

Compound C: anamorelin,
2-amino-N-[1R)-2-[(3R)-3-benzyl-3-(N,N′,N′-trimethylhydrazinocarbonyl)piperidin-1-yl]-1-(1H-indol-3-ylmethyl)-2-oxoethyl]-2-methylpropionamide.

Compound D: ST-1141, another name RC-1141,
(E)-N—((R)-3-([1,1′-biphenyl]-4-yl)-1-(((R)-1-(4-hydroxypiperidin-1-yl)-1-oxo-3-phenylpropan-2-yl)(methyl)amino)-1-oxopropan-2-yl)-4-(1-aminocyclobutyl)-N-methylbut-2-enamide.

Compound E: ulimorelin,
(2R,5S,8R,11R)-5-cyclopropyl-11-(4-fluorobenzyl)-2,7,8-trimethyl-4,5,7,8,10,11,13,14,15,16-decahydro-2H-benzo[q][1,4,7,10,13]oxatetraazacyclooctadecine-6,9,12(3H)-trione.

Compound F: ipamorelin,
(S)-6-amino-2-((R)-2-((R)-2-((S)-2-(2-amino-2-methylpropanamido)-3-(1H-imidazol-5-yl)propanamido)-3-(naphthalen-2-yl)propanamido)-3-phenylpropanamido)hexanamide.

### Example 1

### Measurement of Intra-Gastric pH in Dogs

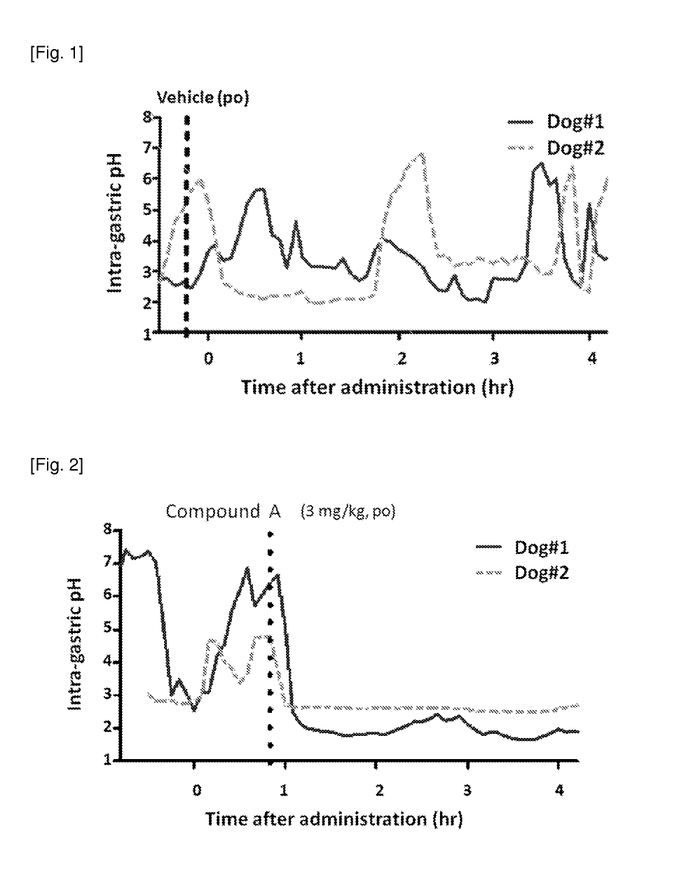

Male beagle dogs are used. A metallic cannula is placed in on the left side of the abdomen at the lowest part of the distal gastric corpus region near the greater curvature by a surgical operation. Intra-gastric pH is measured continuously by a flexible pH electrode inserted via the gastric fistula. Vehicle or drugs are administered orally to the dogs. The results are shown in FIG. 1.

### Example 2

Compound A 3 mg/kg is orally administered to dogs in a similar way described in Example 1. The results are shown in FIG. 2. Intra-gastric pH value is between 2 and 7 in the vehicle-treated conscious dogs. The intra-gastric pH value of both dogs administered Compound A rapidly decreases after the administration and then the resulting low pH value is maintained below about 2.5 for more than 3 hours.

### Example 3

Compound B is orally administered to dogs in a similar way described in Example 1. The intra-gastric pH value of the dogs decreases soon just after dosing and resulting low pH is maintained for more than 3 hours.

### Example 4

Compound C is orally administered to dogs in a similar way described in Example 1. The intra-gastric pH value of the dogs decreases soon just after dosing and resulting low pH is maintained for more than 3 hours.

### Example 5

Compound D is orally administered to dogs in a similar way described in Example 1. The intra-gastric pH value of the dogs decreases soon just after dosing and resulting low pH is maintained for more than 3 hours.

### Example 6

Compound E is orally administered to dogs in a similar way described in Example 1. The intra-gastric pH value of the dogs decreases soon just after dosing and resulting low pH is maintained for more than 3 hours.

### Example 7

Compound E is orally administered to dogs in a similar way described in Example 1. The intra-gastric pH value of the dogs decreases soon just after dosing and resulting low pH is maintained for more than 3 hours.

1. A method for the treatment of achlorhydria, which comprises administering an effective amount of one or more selected from the group consisting of a compound of the formula (I), a racemic-diastereomeric mixture, and optical isomer of the said compound, and the pharmaceutically-acceptable salt and a prodrug thereof to a human or an animal: 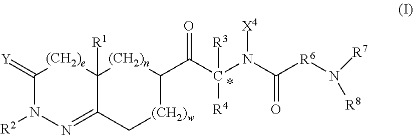
wherein
e is 0 or 1;
n and w are each independently 0, 1 or 2, provided that w and n cannot both be 0 at the same time;
Y is oxygen or sulfur;
R¹ is hydrogen, —CN, —(CH₂)_{q}N(X⁶)C(O)X⁶, —(CH₂)_{q}N(X⁶)C(O)(CH₂)ₜ-A¹, —(CH₂)_{q}N(X⁶)SO₂(CH₂)ₜ—, -A¹, —(CH₂)_{q}N(X⁶)SO₂X⁶, —(CH₂)_{q}N(X⁶)C(O)N(X⁶)(CH₂)ₜ-A¹, —(CH₂)_{q}N(X⁶)C(O)N(X⁶)(X⁶), —(CH₂)_{q}C(O)N(X⁶)(X⁶), —(CH₂)_{q}C(O)N(X⁶)(CH₂)ₜ-A¹, —(CH₂)_{q}C(O)OX⁶, —(CH₂)_{q}C(O)O(CH₂)ₜ-A¹, —(CH₂)_{q}OX⁶, —(CH₂)_{q}OC(O)X⁶, —(CH₂)_{q}OC(O)(CH₂)ₜ-A¹, —(CH₂)_{q}OC(O)N(X⁶)(CH₂)ₜ-A¹, —(CH₂)_{q}OC(O)N(X⁶)(X⁶), —(CH₂)_{q}C(O)X⁶, —(CH₂)_{q}C(O)(CH₂)ₜ-A¹, —(CH₂)_{q}N(X⁶)C(O)OX⁶, —(CH₂)_{q}N(X⁶)SO₂N(X⁶)(X⁶), —(CH₂)_{q}S(O)ₘX⁶, —(CH₂)_{q}S(O)ₘ(CH₂)ₜ-A¹, —(C₁-C₁₀)alkyl, —(CH₂)ₜ-A¹, —(CH₂)_{q}—(C₃-C₇)cycloalkyl, —(CH₂)_{q}—Y¹—(C₁-C₆)alkyl, —(CH₂)_{q}—Y¹—(CH₂)ₜ-A¹ or —(CH₂)_{q}—Y¹—(CH₂)(C₃-C₇)cycloalkyl;
where the alkyl and cycloalkyl groups in the definition of R¹ are optionally substituted with (C₁-C₄)alkyl, hydroxyl, (C₁-C₄)alkoxy, carboxyl, —CONH₂, —S(O)ₘ(C₁-C₆)alkyl, —CO₂(C₁-C₄)alkyl ester, 1H-tetrazol-5-yl or 1, 2 or 3 fluoro;
Y¹ is O, S(O)ₘ, —C(O)NX⁶—, CH═CH—, —C≡C—, —N(X⁶)C(O)—, —C(O)NX⁶—, —C(O)O—, —OC(O)N(X⁶)— or —OC(O)—;
q is 0, 1, 2, 3 or 4;
t is 0, 1, 2 or 3;
m is 0, 1 or 2;
said (CH₂)_{q} group and (CH₂)ₜ group may each be optionally substituted with hydroxyl, (C₁-C₄)alkoxy, carboxyl, —CONH₂, —S(O)ₘ—(C₁-C₆)alkyl, —CO₂(C₁-C₄)alkyl ester, 1H-tetrazol-5-yl, 1, 2 or 3 fluoro, or 1 or 2 (C₁-C₄)alkyl;
R² is hydrogen, (C₁-C₈)alkyl, —(C₀-C₃)alkyl-(C₃-C₈)cycloalkyl, —(C₁-C₄)alkyl-A¹ or A¹;
where the alkyl groups and the cycloalkyl groups in the definition of R² are optionally substituted with hydroxyl, —C(O)OX⁶, —C(O)N(X⁶)(X⁶), —N(X⁶)(X⁶), —S(O)ₘ(C₁-C₆)alkyl, —C(O)A¹, —C(O)(X⁶), CF₃, CN or 1, 2 or 3 halogen;
R³ is A¹, (C₁-C₁₀)alkyl, —(C₁-C₆)alkyl-A¹, —(C₁-C₆)alkyl-(C₃-C₇)cycloalkyl, —(C₁-C₅)alkyl-X¹—(C₁-C₅)alkyl, —(C₁-C₅)alkyl-X¹—(C₀-C₅)alkyl-A¹ or —(C₁-C₅)alkyl-X¹—(C₁-C₅)alkyl-(C₃-C₇)cycloalkyl;
where the alkyl groups in the definition of R³ are optionally substituted with, —S(O)ₘ(C₁-C₆)alkyl, —C(O)OX³, 1, 2, 3, 4 or 5 halogens, or 1, 2 or 3 OX³;
X¹ is O, S(O)ₘ, —N(X²)C(O)—, —C(O)N(X²)—, —OC(O)—, —C(O)O—, —CX²═CX²—, —N(X²)C(O)O—, —OC(O)N(X²)—OR—C≡C—;
R⁴ is hydrogen, (C₁-C₆)alkyl or (C₃-C₇)cycloalkyl, or R⁴ is taken together with R³ and the carbon atom to which they are attached and form (C₅-C₇)cycloalkyl, (C₅-C₇)cycloalkenyl, a partially saturated or fully saturated 4- to 8-membered ring having 1 to 4 heteroatoms independently selected from the group consisting of oxygen, sulfur and nitrogen, or R⁴ is a bicyclic ring system consisting of a partially saturated or fully saturated 5- or 6-membered ring, fused to a partially saturated, fully unsaturated or fully saturated 5- or 6-membered ring, optionally having 1 to 4 heteroatoms independently selected from the group consisting of nitrogen, sulfur and oxygen;
X⁴ is hydrogen or (C₁-C₆)alkyl or X⁴ is taken together with R⁴ and the nitrogen atom to which X⁴ is attached and the carbon atom to which R⁴ is attached and form a five to seven membered ring;
R⁶ is a bond or 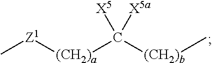
where a and b are independently 0, 1, 2 or 3;
X⁵ and X^{5a} are each independently selected from the group consisting of hydrogen, trifluoromethyl, A¹ and optionally substituted (C₁-C₆)alkyl;
the optionally substituted (C₁-C₆)alkyl in the definition of X⁵ and X^{5a} is optionally substituted with a substituent selected from the group consisting of A¹, OX², —S(O)ₘ(C₁-C₆)alkyl, —C(O)OX², (C₃-C₇)cycloalkyl, —N(X²)(X²) and —C(O)N(X²)(X²);
in which the carbon bearing X⁵ or X^{5a} forms one or two alkylene bridges with the nitrogen atom bearing R⁷ and R⁸ wherein each alkylene bridge contains 1 to 5 carbon atoms, provided that when one alkylene bridge is formed then X⁵ or X^{5a} but not both may be on the carbon atom and R⁷ or R⁸ but not both may be on the nitrogen atom and further provided that when two alkylene bridges are formed then X⁵ and X^{5a} cannot be on the carbon atom and R⁷ and R⁸ cannot be on the nitrogen atom;
or X⁵ is taken together with X^{5a} and the carbon atom to which they are attached and form a partially saturated or fully saturated 3- to 7-membered ring, or a partially saturated or fully saturated 4- to 8-membered ring having 1 to 4 heteroatoms independently selected from the group consisting of oxygen, sulfur and nitrogen,
or X⁵ is taken together with X^{5a} and the carbon atom to which they are attached and form a bicyclic ring system consisting of a partially saturated or fully saturated 5- or 6-membered ring, optionally having 1 or 2 heteroatoms independently selected from the group consisting of nitrogen, sulfur and oxygen, fused to a partially saturated, fully saturated or fully unsaturated 5- or 6-membered ring, optionally having 1 to 4 heteroatoms independently selected from the group consisting of nitrogen, sulfur and oxygen;
Z¹ is a bond, O or N—X², provided that when a and b are both 0 then Z¹ is not N—X² or O;
R⁷ and R⁸ are independently hydrogen or optionally substituted (C₁-C₆)alkyl;
where the optionally substituted (C₁-C₆)alkyl in the definition of R⁷ and R⁸ is optionally independently substituted with A¹, —C(O)O—(C₁-C₆)alkyl, —S(O)ₘ(C₁-C₆)alkyl, 1 to 5 halogens, 1 to 3 hydroxy groups, 1 to 3 —O—C(O)(C₁-C₁₀)alkyl groups or 1 to 3 (C₁-C₆)alkoxy groups; or
R⁷ and R⁸ can be taken together to form —(CH₂)ᵣ-L-(CH₂)ᵣ—;
where L is C(X²)(X²), S(O)ₘ or N(X²);
A¹ for each occurrence is independently (C₅-C₇)cycloalkenyl, phenyl or a substituent formed by eliminating hydrogen form a partially saturated, fully saturated or fully unsaturated 4- to 8-membered ring optionally having 1 to 4 heteroatoms independently selected from the group consisting of oxygen, sulfur and nitrogen, a bicyclic ring system consisting of a partially saturated, fully unsaturated or fully saturated 5- or 6-membered ring, optionally having 1 to 4 heteroatoms independently selected from the group consisting of nitrogen, sulfur and oxygen, fused to a partially saturated, fully saturated or fully unsaturated 5- or 6-membered ring, optionally having 1 to 4 heteroatoms independently selected from the group consisting of nitrogen, sulfur and oxygen;
A¹ for each occurrence is independently optionally substituted, in one or optionally both rings if A¹ is a bicyclic ring system, with up to three substituents, each substituent independently selected from the group consisting of F, Cl, Br, I, OCF₃, OCF₂H, CF₃, CH₃, OCH₃, —OX⁶, —C(O)N(X⁶)(X⁶), —C(O)OX⁶, oxo, (C₁-C₆)alkyl, nitro, cyano, benzyl, —S(O)ₘ(C₁-C₆)alkyl, 1H-tetrazol-5-yl, phenyl, phenoxy, phenylalkyloxy, halophenyl, methylenedioxy, —N(X⁶)(X⁶), —N(X⁶)C(O)(X⁶), —SO₂N(X⁶)(X⁶), —N(X⁶)SO₂-phenyl, —N(X⁶)SO₂X⁶, —CONX¹¹X¹², —SO₂NX¹¹X¹², —NX⁶SO₂X¹², —NX⁶CONX¹¹X¹², —NX⁶SO₂NX¹¹X¹², —NX⁶C(O)X¹², imidazolyl, thiazolyl or tetrazolyl, provided that if A¹ is optionally substituted with methylenedioxy then it can only be substituted with one methylenedioxy;
where X¹¹ is hydrogen or optionally substituted (C₁-C₆)alkyl;
the optionally substituted (C₁-C₆)alkyl defined for X¹¹ is optionally independently substituted with phenyl, phenoxy, (C₁-C₆)alkoxycarbonyl, —S(O)ₘ(C₁-C₆)alkyl, 1 to 5 halogens, 1 to 3 hydroxy, 1 to 3 (C₁-C₁₀)alkanoyloxy or 1 to 3 (C₁-C₆)alkoxy;
X¹² is hydrogen, (C₁-C₆)alkyl, phenyl, thiazolyl, imidazolyl, furyl or thienyl, provided that when X¹² is not hydrogen, X¹² is optionally substituted with one to three substituents independently selected from the group consisting of Cl, F, CH₃, OCH₃, OCF₃ and CF₃;
or X¹¹ and X¹² are taken together to form —(CH₂)ᵣ-L¹-(CH₂)ᵣ—;
where L¹ is C(X²)(X²), O, S(O)ₘ or N(X²);
r for each occurrence is independently 1, 2 or 3;
X² for each occurrence is independently hydrogen, optionally substituted (C₁-C₆)alkyl, or optionally substituted (C₃-C₇)cycloalkyl, where the optionally substituted (C₁-C₆)alkyl and optionally substituted (C₃-C₇)cycloalkyl in the definition of X² are optionally independently substituted with —S(O)ₘ(C₁-C₆)alkyl, —C(O)OX³, 1 to 5 halogens or 1 to 3 OX³;
X³ for each occurrence is independently hydrogen or (C₁-C₆)alkyl;
X⁶ is independently hydrogen, optionally substituted (C₁-C₆)alkyl, (C₂-C₆)halogenated alkyl, optionally substituted (C₃-C₇)cycloalkyl, (C₃-C₇)-halogenatedcycloalkyl, where optionally substituted (C₁-C₆)alkyl and optionally substituted (C₃-C₇)cycloalkyl in the definition of X⁶ is optionally independently substituted by 1 or 2 (C₁-C₄)alkyl, hydroxyl, (C₁-C₄)alkoxy, carboxyl, CONH₂, —S(O)ₘ(C₁-C₆)alkyl, carboxylate, (C₁-C₄)alkyl carboxy ester, or 1H-tetrazol-5-yl; or
when there are two X⁶ groups on one atom and both X⁶ are independently (C₁-C₆)alkyl, the two (C₁-C₆)alkyl groups may be optionally joined and, together with the atom to which the two X⁶ groups are attached, form a 4- to 9-membered ring optionally having oxygen, sulfur or NX⁷;
X⁷ is hydrogen or (C₁-C₆)alkyl optionally substituted with hydroxyl; and m for each occurrence is independently 0, 1 or 2;
with the proviso that:
X⁶ and X¹² cannot be hydrogen when it is attached to C(O) or SO₂ in the form C(O)X⁶, C(O)X¹², SO₂X⁶ or SO₂X¹²;
when R⁶ is a bond then L is N(X²) and each r in the definition —(CH₂)ᵣ-L-(CH₂)ᵣ— is independently 2 or 3; and
C. represents an asymmetric carbon atom.

2. A method for the treatment of achlorhydria, which comprises administering an effective amount of one or more selected from the group consisting of a compound of the formula (II), racemic-diastereomeric mixture, and an optical isomer of the said compound, and a pharmaceutically-acceptable salts and prodrug thereof to a human or animal: 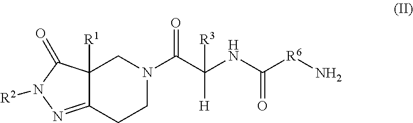
wherein
R¹ is —(C₁-C₃)alkyl-phenyl, —(C₁-C₃)alkyl-pyridyl, —(C₁-C₃)alkyl-quinolyl or —(C₁-C₃)alkyl-thiazolyl, where the phenyl in R¹ is optionally substituted with one or two substituents selected from the group consisting of halo, CF₃, CH₃ and phenyl;
R² is —(C₁-C₄)alkyl or —(C₁-C₄)alkyl-CF₃;
R³ is —(C₁-C₄)alkylindolyl, —(C₁-C₄)alkylphenyl, —(C₁-C₄)alkyl-O—(C₁-C₄)alkyl-Ar, —(C₁-C₄)alkyl-S—(C₁-C₄)alkyl-Ar, where Ar is phenyl, thienyl, thiazolyl, pyridyl, pyrimidinyl or benzisoxazolyl, the said Ar is optionally substituted with one or two substituents selected from the group consisting of halo, OCF₃, CF₃ and CH₃; and
R⁶ is —C(X⁵)(X⁵), where X⁵ is —(C₁-C₆)alkyl.

3. The method according to claim 1, wherein the compound is selected from the group consisting of the following compounds:
2-amino-N-[1-(3a-(R,S)-benzyl-2-methyl-3-oxo-2,3,3a,4,6,7-hexahydropyrazolo[4,3-c]pyridine-5-carbonyl)-4-phenyl(R)-butyl]isobutyramide;
2-amino-N-[1-(3a-(R)-benzyl-2-methyl-3-oxo-2,3,3a,4,6,7-hexahydropyrazolo[4,3-c]pyridine-5-carbonyl)-4-phenyl(R)-butyl]isobutyramide;
2-amino-N-[1-(3a-(S)-benzyl-2-methyl-3-oxo-2,3,3a,4,6,7-hexahydropyrazolo[4,3-c]pyridine-5-carbonyl)-4-phenyl(R)-butyl]isobutyramide;
2-amino-N-[2-(3a-(R,S)-benzyl-2-methyl-3-oxo-2,3,3a,4,6,7-hexahydropyrazolo[4,3-c]pyridin-5-yl)-1-(R)-(1H-indol-3-ylmethyl)-2-oxo-ethyl]isobutyramide;
2-amino-N-[2-(3a-(R)-benzyl-2-methyl-3-oxo-2,3,3a,4,6,7-hexahydropyrazolo[4,3-c]pyridin-5-yl)-1-(R)-(1H-indol-3-ylmethyl)-2-oxo-ethyl]isobutyramide;
2-amino-N-[2-(3a-(S)-benzyl-2-methyl-3-oxo-2,3,3a,4,6,7-hexahydropyrazolo[4,3-c]pyridin-5-yl)-1-(R)-(1H-indol-3-ylmethyl)-2-oxo-ethyl]isobutyramide;
2-amino-N-[2-(3a-(R,S)-benzyl-2-ethyl-3-oxo-2,3,3a,4,6,7-hexahydropyrazolo[4,3-c]pyridin-5-yl)-1-(R)-(1H-indol-3-ylmethyl)-2-oxo-ethyl]isobutyramide;
2-amino-N-[2-(3a-(R)-benzyl-2-ethyl-3-oxo-2,3,3a,4,6,7-hexahydropyrazolo[4,3-c]pyridin-5-yl)-1-(R)-(1H-indol-3-ylmethyl)-2-oxo-ethyl]isobutyramide;
2-amino-N-[2-(3a-(S)-benzyl-2-ethyl-3-oxo-2,3,3a,4,6,7-hexahydropyrazolo[4,3-c]pyridin-5-yl)-1-(R)-(1H-indol-3-ylmethyl)-2-oxo-ethyl]isobutyramide;
2-amino-N-[2-(3a-(R,S)-(4-fluoro-benzyl)-2-methyl-3-oxo-2,3,3a,4,6,7-hexahydropyrazolo[4,3-c]pyridin-5-yl)-1-(R)-(1H-indol-3-ylmethyl)-2-oxo-ethyl]isobutyramide;
2-amino-N-[2-(3a-(R)-(4-fluoro-benzyl)-2-methyl-3-oxo-2,3,3a,4,6,7-hexahydropyrazolo[4,3-c]pyridin-5-yl)-1-(R)-(1H-indol-3-ylmethyl)-2-oxo-ethyl]isobutyramide;
2-amino-N-[2-(3a-(S)-(4-fluoro-benzyl)-2-methyl-3-oxo-2,3,3a,4,6,7-hexahydropyrazolo[4,3-c]pyridin-5-yl)-1-(R)-(1H-indol-3-ylmethyl)-2-oxo-ethyl]isobutyramide;
2-amino-N-[2-(3a-(R,S)-benzyl-2-methyl-3-oxo-2,3,3a,4,6,7-hexahydropyrazolo[4,3-c]pyridin-5-yl)-1-(R)-benzyloxymethyl-2-oxo-ethyl]isobutyramide;
2-amino-N-[2-(3a-(R)-benzyl-2-methyl-3-oxo-2,3,3a,4,6,7-hexahydropyrazolo[4,3-c]pyridin-5-yl)-1-(R)-benzyloxymethyl-2-oxo-ethyl]isobutyramide;
2-amino-N-[2-(3a-(S)-benzyl-2-methyl-3-oxo-2,3,3a,4,6,7-hexahydropyrazolo[4,3-c]pyridin-5-yl)-1-(R)-benzyloxymethyl-2-oxo-ethyl]isobutyramide;
2-amino-N-[2-(3a-(R,S)-benzyl-2-ethyl-3-oxo-2,3,3a,4,6,7-hexahydropyrazolo[4,3-c]pyridin-5-yl)-1-(R)-benzyloxymethyl-2-oxo-ethyl]isobutyramide;
2-amino-N-[2-(3a-(R)-benzyl-2-ethyl-3-oxo-2,3,3a,4,6,7-hexahydropyrazolo[4,3-c]pyridin-5-yl)-1-(R)-benzyloxymethyl-2-oxo-ethyl]isobutyramide;
2-amino-N-[2-(3a-(S)-benzyl-2-ethyl-3-oxo-2,3,3a,4,6,7-hexahydropyrazolo[4,3-c]pyridin-5-yl)-1-(R)-benzyloxymethyl-2-oxo-ethyl]isobutyramide;
2-amino-N-[2-(3a-(R,S)-benzyl-3-oxo-2-(2,2,2-trifluoro-ethyl)-2,3,3a,4,6,7-hexahydropyrazolo[4,3-c]pyridin-5-yl)-1-(R)-benzyloxymethyl-2-oxo-ethyl]isobutyramide;
2-amino-N-[2-(3a-(R)-benzyl-3-oxo-2-(2,2,2-trifluoro-ethyl)-2,3,3a,4,6,7-hexahydropyrazolo[4,3-c]pyridin-5-yl)-1-(R)-benzyloxymethyl-2-oxo-ethyl]isobutyramide;
2-amino-N-[2-(3a-(S)-benzyl-3-oxo-2-(2,2,2-trifluoro-ethyl)-2,3,3a,4,6,7-hexahydropyrazolo[4,3-c]pyridin-5-yl)-1-(R)-benzyloxymethyl-2-oxo-ethyl]isobutyramide;
2-amino-N-[1-(R)-benzyloxymethyl-2-(3a-(R,S)-(4-fluoro-benzyl)-2-methyl-3-oxo-2,3,3a,4,6,7-hexahydropyrazolo[4,3-c]pyridin-5-yl)-2-oxo-ethyl]isobutyramide;
2-amino-N-[1-(R)-benzyloxymethyl-2-(3a-(R)-(4-fluoro-benzyl)-2-methyl-3-oxo-2,3,3a,4,6,7-hexahydropyrazolo[4,3-c]pyridin-5-yl)-2-oxo-ethyl]isobutyramide;
2-amino-N-[1-(R)-benzyloxymethyl-2-(3a-(S)-(4-fluoro-benzyl)-2-methyl-3-oxo-2,3,3a,4,6,7-hexahydropyrazolo[4,3-c]pyridin-5-yl)-2-oxo-ethyl]isobutyramide;
2-amino-N-[2-(3a-(R,S)-benzyl-2-tert-butyl-3-oxo-2,3,3a,4,6,7-hexahydropyrazolo[4,3-c]pyridin-5-yl)-1-(R)-benzyloxymethyl-2-oxo-ethyl]isobutyramide;
2-amino-N-[2-(3a-(R)-benzyl-2-tert-butyl-3-oxo-2,3,3a,4,6,7-hexahydropyrazolo[4,3-c]pyridin-5-yl)-1-(R)-benzyloxymethyl-2-oxo-ethyl]isobutyramide;
2-amino-N-[2-(3a-(S)-benzyl-2-tert-butyl-3-oxo-2,3,3a,4,6,7-hexahydropyrazolo[4,3-c]pyridin-5-yl)-1-(R)-benzyloxymethyl-2-oxo-ethyl]isobutyramide;
2-amino-N-[2-(3a-(R,S)-benzyl-3-oxo-2,3,3a,4,6,7-hexahydropyrazolo[4,3-c]pyridin-5-yl)-1-(R)-benzyloxymethyl-2-oxo-ethyl]isobutyramide;
2-amino-N-[2-(3a-(R)-benzyl-3-oxo-2,3,3a,4,6,7-hexahydropyrazolo[4,3-c]pyridin-5-yl)-1-(R)-benzyloxymethyl-2-oxo-ethyl]isobutyramide;
2-amino-N-[2-(3a-(S)-benzyl-3-oxo-2,3,3a,4,6,7-hexahydropyrazolo[4,3-c]pyridin-5-yl)-1-(R)-benzyloxymethyl-2-oxo-ethyl]isobutyramide;
2-amino-N-[1-(R)-benzyloxymethyl-2-(2-methyl-3-oxo-3a-(R,S)-pyridin-2-ylmethyl-2,3,3a,4,6,7-hexahydropyrazolo[4,3-c]pyridin-5-yl)-2-oxo-ethyl]-2-methyl-propionamide;
2-amino-N-[1-(R)-benzyloxymethyl-2-(2-methyl-3-oxo-3a-(R)-pyridin-2-ylmethyl-2,3,3a,4,6,7-hexahydropyrazolo[4,3-c]pyridin-5-yl)-2-oxo-ethyl]-2-methyl-propionamide;
2-amino-N-[1-(R)-benzyloxymethyl-2-(2-methyl-3-oxo-3a-(S)-pyridin-2-ylmethyl-2,3,3a,4,6,7-hexahydropyrazolo[4,3-c]pyridin-5-yl)-2-oxo-ethyl]-2-methyl-propionamide;
2-amino-N-[1-(R)-(3-chloro-benzyloxymethyl)-2-oxo-2-(3-oxo-3a-(R,S)-pyridin-2-ylmethyl-2-(2,2,2-trifluoro-ethyl)-2,3,3a,4,6,7-hexahydropyrazolo[4,3-c]pyridin-5-yl)-ethyl]-2-methyl-propionamide;
2-amino-N-[1-(R)-(3-chloro-benzyloxymethyl)-2-oxo-2-(3-oxo-3a-(R)-pyridin-2-ylmethyl-2-(2,2,2-trifluoro-ethyl)-2,3,3a,4,6,7-hexahydropyrazolo[4,3-c]pyridin-5-yl)-ethyl]-2-methyl-propionamide;
2-amino-N-[1-(R)-(3-chloro-benzyloxymethyl)-2-oxo-2-(3-oxo-3a-(S)-pyridin-2-ylmethyl-2-(2,2,2-trifluoro-ethyl)-2,3,3a,4,6,7-hexahydropyrazolo[4,3-c]pyridin-5-yl)-ethyl]-2-methyl-propionamide;
2-amino-N-[1-(R)-(4-chloro-benzyloxymethyl)-2-oxo-2-(3-oxo-3a-(R,S)-pyridin-2-ylmethyl-2-(2,2,2-trifluoro-ethyl)-2,3,3a,4,6,7-hexahydropyrazolo[4,3-c]pyridin-5-yl)-ethyl]-2-methyl-propionamide;
2-amino-N-[1-(R)-(4-chloro-benzyloxymethyl)-2-oxo-2-(3-oxo-3a-(R)-pyridin-2-ylmethyl-2-(2,2,2-trifluoro-ethyl)-2,3,3a,4,6,7-hexahydropyrazolo[4,3-c]pyridin-5-yl)-ethyl]-2-methyl-propionamide;
2-amino-N-[1-(R)-(4-chloro-benzyloxymethyl)-2-oxo-2-(3-oxo-3a-(S)-pyridin-2-ylmethyl-2-(2,2,2-trifluoro-ethyl)-2,3,3a,4,6,7-hexahydropyrazolo[4,3-c]pyridin-5-yl)-ethyl]-2-methyl-propionamide;
2-amino-N-[1-(R)-(2,4-dichloro-benzyloxymethyl)-2-oxo-2-(3-oxo-3a-(R,S)-pyridin-2-ylmethyl-2-(2,2,2-trifluoro-ethyl)-2,3,3a,4,6,7-hexahydropyrazolo[4,3-c]pyridin-5-yl)-ethyl]-2-methyl-propionamide;
2-amino-N-[1-(R)-(2,4-dichloro-benzyloxymethyl)-2-oxo-2-(3-oxo-3a-(R)-pyridin-2-ylmethyl-2-(2,2,2-trifluoro-ethyl)-2,3,3a,4,6,7-hexahydropyrazolo[4,3-c]pyridin-5-yl)-ethyl]-2-methyl-propionamide;
2-amino-N-[1-(R)-(2,4-dichloro-benzyloxymethyl)-2-oxo-2-(3-oxo-3a-(S)-pyridin-2-ylmethyl-2-(2,2,2-trifluoro-ethyl)-2,3,3a,4,6,7-hexahydropyrazolo[4,3-c]pyridin-5-yl)-ethyl]-2-methyl-propionamide;
2-amino-N-[1-(R)-(4-chloro-thiophen-2-ylmethoxymethyl)-2-oxo-2-(3-oxo-3a-(R,S)-pyridin-2-ylmethyl-2-(2,2,2-trifluoro-ethyl)-2,3,3a,4,5,7-hexahydropyrazolo[3,4-c]pyridin-6-yl)-ethyl]-2-methyl-propionamide;
2-amino-N-[1-(R)-(4-chloro-thiophen-2-ylmethoxymethyl)-2-oxo-2-(3-oxo-3a-(R)-pyridin-2-ylmethyl-2-(2,2,2-trifluoro-ethyl)-2,3,3a,4,5,7-hexahydropyrazolo[3,4-c]pyridin-6-yl)-ethyl]-2-methyl-propionamide;
2-amino-N-[1-(R)-(4-chloro-thiophen-2-ylmethoxymethyl)-2-oxo-2-(3-oxo-3a-(S)-pyridin-2-ylmethyl-2-(2,2,2-trifluoro-ethyl)-2,3,3a,4,5,7-hexahydropyrazolo[3,4-c]pyridin-6-yl)-ethyl]-2-methyl-propionamide;
2-amino-N-[1-(R)-(2,4-difluoro-benzyloxymethyl)-2-oxo-2-(3-oxo-3a-(R,S)-pyridin-2-ylmethyl-2-(2,2,2-trifluoro-ethyl)-2,3,3a,4,6,7-hexahydropyrazolo[4,3-c]pyridin-5-yl)-ethyl]-2-methyl-propionamide;
2-amino-N-[1-(R)-(2,4-difluoro-benzyloxymethyl)-2-oxo-2-(3-oxo-3a-(R)-pyridin-2-ylmethyl-2-(2,2,2-trifluoro-ethyl)-2,3,3a,4,6,7-hexahydropyrazolo[4,3-c]pyridin-5-yl)-ethyl]-2-methyl-propionamide;
2-amino-N-[1-(R)-(2,4-difluoro-benzyloxymethyl)-2-oxo-2-(3-oxo-3a-(S)-pyridin-2-ylmethyl-2-(2,2,2-trifluoro-ethyl)-2,3,3a,4,6,7-hexahydropyrazolo[4,3-c]pyridin-5-yl)-ethyl]-2-methyl-propionamide;
2-amino-N-[2-(3a-(R,S)-benzyl-2-methyl-3-oxo-2,3,3a,4,6,7-hexahydropyrazolo[4,3-c]pyridin-5-yl)-1-(R)-(3,4-difluoro-benzyloxymethyl)-2-oxo-ethyl]-2-methyl-propionamide;
2-amino-N-[2-(3a-(R)-benzyl-2-methyl-3-oxo-2,3,3a,4,6,7-hexahydropyrazolo[4,3-c]pyridin-5-yl)-1-(R)-(3,4-difluoro-benzyloxymethyl)-2-oxo-ethyl]-2-methyl-propionamide; and
2-amino-N-[2-(3a-(S)-benzyl-2-methyl-3-oxo-2,3,3a,4,6,7-hexahydropyrazolo[4,3-c]pyridin-5-yl)-1-(R)-(3,4-difluoro-benzyloxymethyl)-2-oxo-ethyl]-2-methyl-propionamide;
or a pharmaceutically acceptable salt thereof.

4. The method according to claim 1 or 2, wherein the compound is selected from the group consisting of the following compounds:
2-amino-N-[2-(3a-(R)-benzyl-2-methyl-3-oxo-2,3,3a,4,6,7-hexahydropyrazolo[4,3-c]pyridin-5-yl)-1-(R)-benzyloxymethyl-2-oxo-ethyl]isobutyramide 2-amino-N-[1-(R)-(2,4-difluoro-benzyloxymethyl)-2-oxo-2-(3-oxo-3a-(R)-pyridin-2-ylmethyl-2-(2,2,2-trifluoro-ethyl)-2,3,3a,4,6,7-hexahydropyrazolo[4,3-c]pyridin-5-yl)-ethyl]-2-methyl-propionamide;
and a pharmaceutically acceptable salt thereof.

5. A method for the treatment of achlorhydria, which comprises administering an effective amount of one or more selected from the group consisting of a compound of the formula (III), racemic-diastereomeric mixture, and an optical isomer of the said compound, and pharmaceutically-acceptable salt and prodrug thereof to a human or an animal: 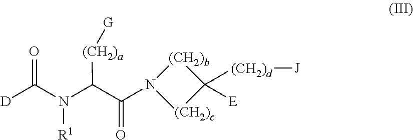
wherein
R¹ is hydrogen, or C₁₋₆-alkyl optionally substituted with one or more aryl or hetaryl;
a and d are independently of each other 0, 1, 2 or 3;
b and c are independently of each other 0, 1, 2, 3, 4 or 5, provided that b+c is 3, 4 or 5,
D is
R²—NH—(CR³R⁴)ₑ—(CH₂)_{f}-M-(CHR⁵)_{q}—(CH₂)ₕ—
wherein R², R³, R⁴ and R⁵ are independently hydrogen or C₁₋₆-alkyl optionally substituted with one or more halogen, amino, hydroxyl, aryl or hetaryl; or
R² and R³ or R² and R⁴ or R³ and R⁴ may optionally form —(CH₂)ᵢ—U—(CH₂)ⱼ—, wherein i and j are independently 1 or 2 and U is —O—, —S— or a bond;
h and f are independently 0, 1, 2, or 3;
g and e are independently 0 or 1;
M is a bond, —CR⁶═CR⁷—, arylene, hetarylene, —O— or —S—;
R⁶ and R⁷ are independently hydrogen, or C₁₋₆-alkyl optionally substituted with one or more aryl or hetaryl;
G is —O—(CH₂)ₖ—R⁸, 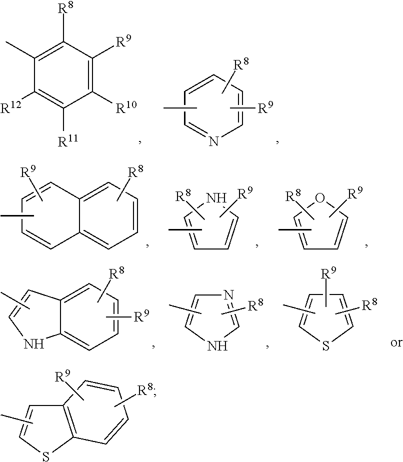
J is —O—(CH₂)₁R¹³, 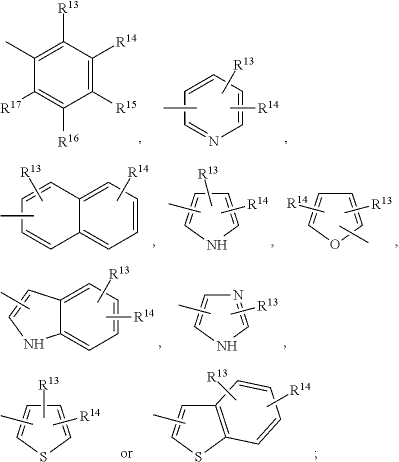
wherein R⁸, R⁹, R¹⁰, R¹¹, R¹², R¹³, R¹⁴, R¹⁵, R¹⁶ and R¹⁷ independently of each other are hydrogen, halogen, aryl, hetaryl, C₁₋₆-alkyl or C₁₋₆-alkoxy;
k and I are independently 0, 1 or 2;
E is —CONR¹⁸R¹⁹, —COOR¹⁹, —(CH₂)ₘ—NR¹⁸SO₂R²⁰, —(CH₂)ₘ—NR¹⁸COR²⁰, —(CH₂)ₘ—OR¹⁹, —(CH₂)ₘ—OCOR²⁰, —CH(R¹⁸)R¹⁹, —(CH₂)ₘ—NR¹⁸—CS—NR¹⁹R²¹; or
—(CH₂)ₘ—NR¹⁸—CO—NR¹⁹R²¹; or
E is —CONR²²NR²³R²⁴;
wherein R²² is hydrogen, C₁₋₆-alkyl optionally substituted with one or more aryl or hetaryl, or aryl or hetaryl optionally substituted with one or more C₁₋₆-alkyl; R²³ is C₁₋₆-alkyl optionally substituted with one or more aryl or hetaryl, or C₁₋₇-acyl; and R²⁴ is hydrogen, C₁₋₆-alkyl optionally substituted with one or more aryl or hetaryl; or aryl or hetaryl optionally substituted with one or more C₁₋₆-alkyl; or
R²² and R²³ together with the nitrogen atoms to which they are attached may form a heterocyclic system optionally substituted with one or more C₁₋₆-alkyl, halogen, amino, hydroxyl, aryl or hetaryl; or
R²² and R²⁴ together with the nitrogen atoms to which they are attached may form a heterocyclic system optionally substituted with one or more C₁₋₆-alkyl, halogen, amino, hydroxyl, aryl or hetaryl; or
R²³ and R²⁴ together with the nitrogen atom to which they are attached may form a heterocyclic system optionally substituted with one or more C₁₋₆-alkyl, halogen, amino, hydroxyl, aryl or hetaryl;
wherein m is 0, 1, 2 or 3,
R¹⁸, R¹⁹ and R²¹ independently are hydrogen or C₁₋₆-alkyl optionally substituted with halogen, —N(R²⁵)R²⁶, wherein R²⁵ and R²⁶ are independently hydrogen or C₁₋₆-alkyl; hydroxyl, C₁₋₆-alkoxy, C₁₋₆-alkoxycarbonyl, C₁₋₆-alkylcarbonyloxy or aryl;
or R¹⁹ is 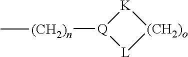
wherein
Q is —CH< or —N<,
K and L are independently —CH₂, —CO—, —O—, —S—, —NR²⁷— or a bond,
where R²⁷ is hydrogen or C₁₋₆-alkyl;
n and o are independently 0, 1, 2, 3 or 4;
R²⁰ is C₁₋₆-alkyl, aryl or hetaryl;
or a pharmaceutically acceptable salt thereof;
with the proviso that
if M is a bond then E is —CONR²²NR²³R²⁴.

6. The method according to claim 5, wherein the compound is following formula (IV):

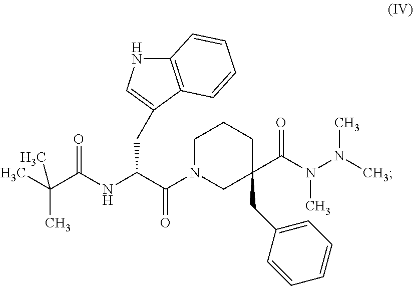 [SMILES: CN(C)N(C)C(=O)[C@@]1(Cc2ccccc2)CCCN(C(=O)[C@@H](Cc2c[nH]c3ccccc23)NC(=O)C(C)(C)C)C1]

7. A method for the treatment of achlorhydria, which comprises administering an effective amount of one or more selected from the group consisting of a compound of the formula (V), racemic-diastereomeric mixture, and an optical isomer of the said compound, and a pharmaceutically-acceptable salt and prodrug thereof to a human or an animal: 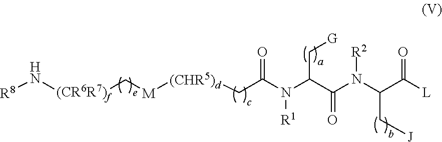
wherein
R¹ is hydrogen or C₁₋₆-alkyl;
R² is hydrogen or C₁₋₆-alkyl;
L is 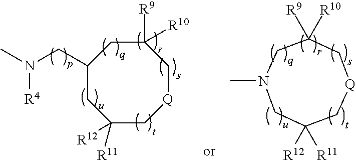
wherein R⁴ is hydrogen or C₁₋₆-alkyl;
p is 0 or 1;
q, s, t, u are independently from each other 0, 1, 2, 3 or 4;
r is 0 or 1;
the sum q+r+s+t+u is 0, 1, 2, 3, or 4;
R⁹, R¹⁰, R¹¹, and R¹² are independently from each other hydrogen or C₁₋₆-alkyl;
Q is >N—R¹³ or 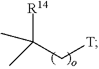
wherein o is 0, 1 or 2;
T is —N(R¹⁵)(R¹⁶) or hydroxyl;
R¹³, R¹⁵, and R¹⁶ are independently from each other hydrogen or C₁₋₆-alkyl;
R¹⁴ is hydrogen, aryl or hetaryl;
G is —O—(CH₂)ₖ—R¹⁷, 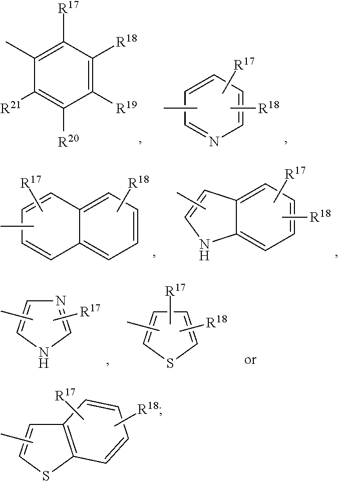
wherein R¹⁷, R¹⁸, R¹⁹, R²⁰ and R²¹ independently from each other are hydrogen, halogen, aryl, hetaryl, C₁₋₆-alkyl or C₁₋₆-alkoxy;
k is 0, 1 or 2;
J is —O—(CH₂)ₗR²², 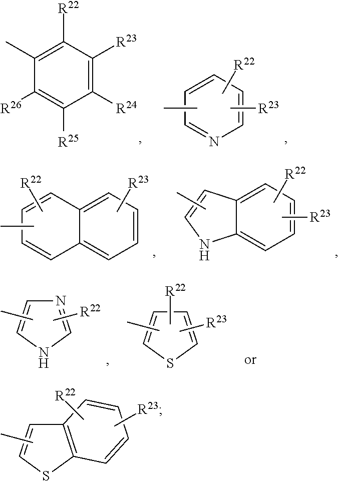
wherein R²², R²³, R²⁴, R²⁵ and R²⁶ independently from each other are hydrogen, halogen, aryl, hetaryl, C₁₋₆-alkyl or C₁₋₆-alkoxy;
I is 0, 1 or 2;
a is 0, 1, or 2;
b is 0, 1, or 2;
c is 0, 1, or 2;
d is 0 or 1;
e is 0, 1, 2, or 3;
f is 0 or 1;
R⁵ is hydrogen or C₁₋₆-alkyl optionally substituted with one or more hydroxyl, aryl or hetaryl;
R⁶ and R⁷ are independently from each other hydrogen or C₁₋₆-alkyl, optionally substituted with one or more halogen, amino, hydroxyl, aryl, or hetaryl;
R⁸ is hydrogen or C₁₋₆-alkyl, optionally substituted with one or more halogen, amino, hydroxyl, aryl, or hetaryl;
R⁸ and R⁷ or R⁶ and R⁸ or R⁷ and R⁸ may optionally form —(CH₂)ᵢ—U—(CH₂)ⱼ—, wherein i and j independently from each other are 1, 2 or 3 and U is —O—, —S—, or a bond;
M is arylene, hetarylene, —O—, —S— or —CR²⁷═CR²⁸—;
R²⁷ and R²⁸ are independently from each other hydrogen or C₁₋₆-alkyl, optionally substituted with one or more aryl or hetaryl.

8. The method according to claim 7, wherein the compound is following formula (VI):

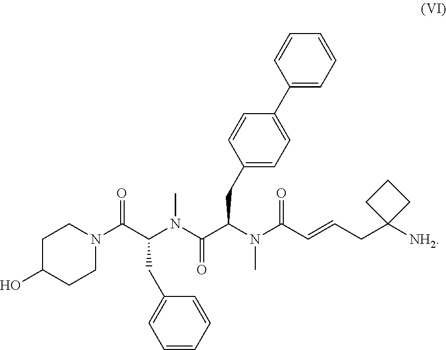 [SMILES: CN(C(=O)/C=C/CC1(N)CCC1)[C@H](Cc1ccc(-c2ccccc2)cc1)C(=O)N(C)[C@H](Cc1ccccc1)C(=O)N1CCC(O)CC1]

9. A method for the treatment of achlorhydria, which comprises administering and effective amount of one or more selected from the group consisting of a compound of the formula (VII), a racemic-diastereomeric mixture, and an optical isomer of the said compound, and a pharmaceutically-acceptable salt and prodrug thereof to a human or an animal: 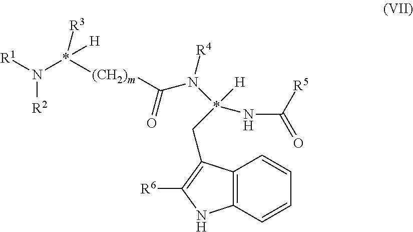
wherein * means a carbon atom which, when a chiral carbon atom, has a R or S configuration, one of R¹ and R³ is an hydrogen atom and the other is a group of formula (A) 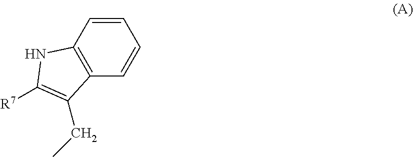
R² is a hydrogen atom, a linear or branched C₁-C₆ alkyl group, an aryl group, a heterocyclic group, a cycloalkyl group, a (CH₂)ₙ-aryl group, a (CH₂)ₙ-heterocyclic group, a (CH₂)ₙ-cycloalkyl group, a methylsulfonyl group, a phenylsulfonyl group, a C(O)R⁸ group or a group according to one of formulas (B) to (G): 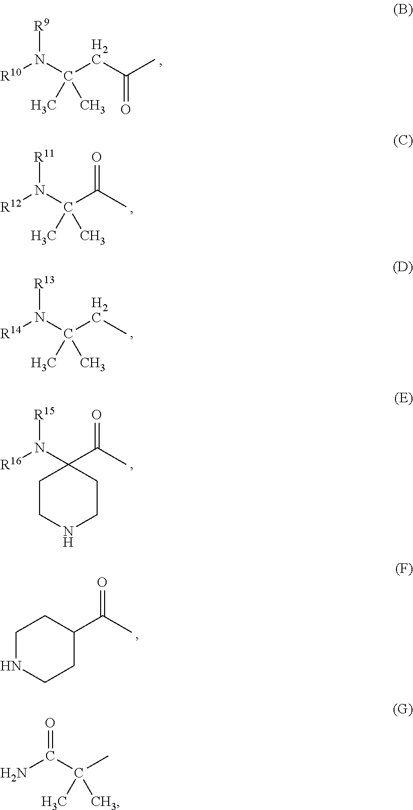
R⁴ is a hydrogen atom or a linear or branched C₁-C₄-alkyl group,
R⁵ is a hydrogen atom, a linear or branched C₁-C₄-alkyl group, a (CH₂)ₙ-aryl group, a (CH₂)ₙ-heterocyclic group, a (CH₂)ₙ-cycloalkyl group or an amino group,
R⁶ and R⁷ are independently from each other a hydrogen atom or a linear or branched C₁-C₄-alkyl group,
R⁸ is a linear or branched C₁-C₆-alkyl group,
R⁹, R¹⁰, R¹¹, R¹², R¹³, R¹⁴, R¹⁵, and R¹⁶ are independently from each other a hydrogen atom or a linear or branched C₁-C₄-alkyl group,
m is 0, 1 or 2 and n is 1 or 2.

10. The method according to claim 9, wherein the compound is following formula (VIII):

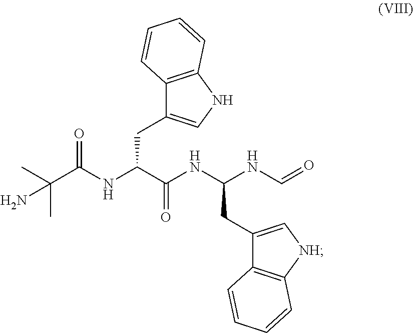 [SMILES: CC(C)(N)C(=O)N[C@H](Cc1c[nH]c2ccccc12)C(=O)N[C@H](Cc1c[nH]c2ccccc12)NC=O]

11. A method for the treatment of achlorhydria, which comprises administering an effective amount of one or more selected from the group consisting of a compound of the formula (IX), a racemic-diastereomeric mixture, and an optical isomer of the said compound, and a pharmaceutically-acceptable salt and a prodrug thereof to a human or an animal: 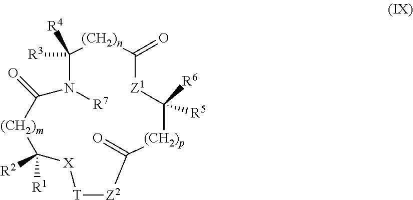
wherein:
R¹ is hydrogen or a side chain of an amino acid, or alternatively R¹ and R² together form a 4-, 5-, 6-, 7- or 8-membered ring, optionally comprising an O, S or N atom in the ring, wherein the ring is optionally substituted with R⁸ as defined below, or alternatively R¹ and R⁹ together form a 3-, 4-, 5-, 6- or 7-membered ring, optionally comprising an O, S or additional N atom in the ring, wherein the ring is optionally substituted with R⁸ as defined below;
R² is hydrogen or a side chain of an amino acid, or alternatively R¹ and R² together form a 4-, 5-, 6-, 7- or 8-membered ring, optionally comprising an O, S or N atom in the ring, wherein the ring is optionally substituted with R⁸ as defined below; or alternatively R² and R⁹ together form a 3-, 4-, 5-, 6- or 7-membered ring, optionally comprising an O, S or additional N atom in the ring, wherein the ring is optionally substituted with R⁸ as defined below;
R³ is hydrogen or a side chain of an amino acid, or alternatively R³ and R⁴ together form a 3-, 4-, 5-, 6- or 7-membered ring, optionally comprising an O or S atom in the ring, wherein the ring is optionally substituted with R⁸ as defined below, or alternatively R³ and R⁷ or R³ and R¹¹ together form a 4-, 5-, 6-, 7- or 8-membered heterocyclic ring, optionally comprising an O, S or additional N atom in the ring, wherein the ring is optionally substituted with R⁸ as defined below;
R⁴ is hydrogen or a side chain of an amino acid, or alternatively R³ and R⁴ together form a 3-, 4-, 5-, 6- or 7-membered ring, optionally comprising an O or S atom in the ring, wherein the ring is optionally substituted with R⁸ as defined below, or alternatively R⁴ and R⁷ or R⁴ and R¹¹ together form a 4-, 5-, 6-, 7- or 8-membered heterocyclic ring, optionally comprising an O, S or additional N atom in the ring, wherein the ring is optionally substituted with R⁸ as defined below;
R⁵ and R⁶ are each independently hydrogen or a side chain of an amino acid or alternatively, R⁵ and R⁶ together form a 3-, 4-, 5-, 6- or 7-membered ring, optionally comprising an O, S or N atom in the ring, wherein the ring is optionally substituted with R⁸ as defined below;
R⁷ is hydrogen, C₁-C₁₀-alkyl, substituted C₁-C₁₀-alkyl, cycloalkyl, substituted cycloalkyl, a heterocyclic group, a substituted heterocyclic group, or alternatively R³ and R⁷ or R⁴ and R⁷, together form a 4-, 5-, 6-, 7- or 8-membered heterocyclic ring optionally comprising an O, S or additional N atom in the ring, wherein the ring is optionally substituted with R⁸;
R⁸ is substituted for one or more hydrogen atoms on a 3-, 4-, 5-, 6-, 7- or 8-membered ring structure and is independently selected from the group consisting of alkyl, substituted alkyl, cycloalkyl, substituted cycloalkyl, a heterocyclic group, a substituted heterocyclic group, aryl, substituted aryl, heteroaryl, substituted heteroaryl, hydroxy, alkoxy, aryloxy, oxo, amino, halogen, formyl, acyl, carboxy, carboxyalkyl, carboxyaryl, amido, carbamoyl, guanidino, ureido, amidino, mercapto, sulfinyl, sulfonyl and sulfonamido, or, alternatively, R⁸ is a fused cycloalkyl, a substituted fused cycloalkyl, a fused heterocyclic group, a substituted fused heterocyclic group, a fused aryl, a substituted fused aryl, a fused heteroaryl or a substituted fused heteroaryl;
X is O, NR⁹ or N(R¹⁰)₂⁺;
wherein R⁹ is hydrogen, C₁-C₁₀-alkyl, substituted C₁-C₁₀-alkyl, sulfonyl, sulfonamido or amidino, and R¹⁰ is hydrogen, C₁-C₁₀-alkyl, or substituted C₁-C₁₀-alkyl, or alternatively R⁹ and R¹ together form a 3-, 4-, 5-, 6- or 7-membered ring, optionally comprising an O, S or additional N atom in the ring, wherein the ring is optionally substituted with R⁸ as defined previously;
Z¹ is O or NR¹¹;
wherein R¹¹ is hydrogen, C₁-C₁₀-alkyl, or substituted C₁-C₁₀-alkyl, or alternatively R³ and R¹¹ or R⁴ and R¹¹ together form a 4-, 5-, 6-, 7- or 8-membered heterocyclic ring, optionally comprising an O, S or additional N atom in the ring, wherein the ring is optionally substituted with R⁸ as defined above;
Z² is O or NR¹²,
wherein R¹² is hydrogen, C₁-C₁₀-alkyl, or substituted C₁-C₁₀-alkyl;
m, n and p are each independently 0, 1 or 2;
T is a bivalent radical of formula
—U—(CH₂)_{d}—W—Y—Z—(CH₂)ₑ—,
wherein d and e are each independently 0, 1, 2, 3, 4 or 5; Y and Z are each optionally present; U is —CR²¹R²²—, or —C(═O)— and is bonded to X of formula (IX); W, Y and Z are each independently selected from the group consisting of —O—, —NR²³—, —S—, —SO—, —SO₂—, —C(═O)—O—, —O—C(═O)—, —C(═O)—NH—, —NH—C(═O)—, —SO₂— NH—, —NH—SO₂—, —CR²⁴R²⁵—, —CH═CH— with the configuration Z or E, —C≡C— and the ring structures below: 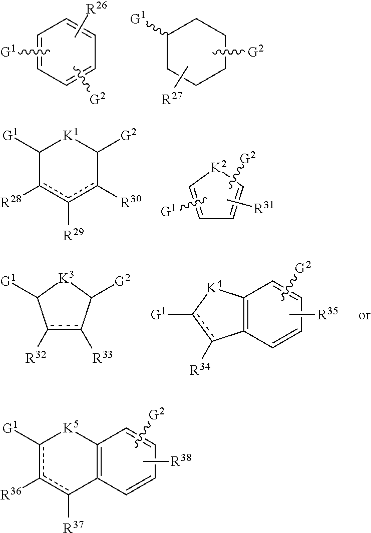
wherein G¹ and G² are each independently a bond or a bivalent radical selected from the group consisting of —O—, —NR³⁹—, —S—, —SO—, —SO₂—, —C(═O)—, —C(═O)—O—, —O—C(═O)—, —C(═O)NH—, —NH—C(═O)—, —SO₂—NH—, —NH—SO₂—, —CR⁴⁰R⁴¹—, —CH═CH— with the configuration Z or E, and —C≡C—; with G¹ being bonded closest to the group U; wherein any carbon atom in the rings not otherwise defined, is optionally replaced by N, with the proviso that the ring cannot contain more than four N atoms; K¹, K², K³, K⁴ and K⁵ are each independently O, NR⁴² or S, wherein R⁴² is as defined below;
R²¹ and R²² are each independently hydrogen, C₁-C₁₀-alkyl, or substituted C₁-C₁₀-alkyl, or alternatively R²¹ and R²² together form a 3- to 12-membered cyclic ring optionally comprising one or more heteroatoms selected from the group consisting of O, S and N, wherein the ring is optionally substituted with R⁸ as defined previously;
R²³, R³⁹ and R⁴² are each independently hydrogen, alkyl, substituted alkyl, cycloalkyl, substituted cycloalkyl, a heterocyclic group, a substituted heterocyclic group, aryl, substituted aryl, heteroaryl, substituted heteroaryl, formyl, acyl, carboxyalkyl, carboxyaryl, amido, amidino, sulfonyl or sulfonamido;
R²⁴ and R²⁵ are each independently hydrogen, C₁-C₁₀-alkyl, substituted C₁-C₁₀-alkyl, R^{AA}, wherein R^{AA} is a side chain of an amino acid, or alternatively R²⁴ and R²⁵ together form a 3- to 12-membered cyclic ring optionally comprising one or more heteroatoms selected from the group consisting of O, S and N; or alternatively one of R²⁴ and R²⁵ is hydroxy, alkoxy, aryloxy, amino, mercapto, carbamoyl, amidino, ureido or guanidino while the other is hydrogen, C₁-C₁₀-alkyl or substituted C₁-C₁₀-alkyl, except when the carbon to which R²⁴ and R²⁵ are bonded is also bonded to another heteroatom;
R²⁶, R³¹, R³⁵ and R³⁸ are each optionally present and, when present, are substituted for one or more hydrogen atoms on the indicated ring and each is independently selected from the group consisting of halogen, trifluoromethyl, alkyl, substituted alkyl, cycloalkyl, substituted cycloalkyl, a heterocyclic group, a substituted heterocyclic group, aryl, substituted aryl, heteroaryl, substituted heteroaryl, hydroxy, alkoxy, aryloxy, amino, formyl, acyl, carboxy, carboxyalkyl, carboxyaryl, amido, carbamoyl, guanidino, ureido, amidino, cyano, nitro, mercapto, sulfinyl, sulfonyl and sulfonamido;
R²⁷ is optionally present and, when present, is substituted for one or more hydrogen atoms on the indicated ring and each is independently selected from the group consisting of alkyl, substituted alkyl, cycloalkyl, substituted cycloalkyl, a heterocyclic group, a substituted heterocyclic group, aryl, substituted aryl, heteroaryl, substituted heteroaryl, hydroxy, alkoxy, aryloxy, oxo, amino, formyl, acyl, carboxy, carboxyalkyl, carboxyaryl, amido, carbamoyl, guanidino, ureido, amidino, mercapto, sulfinyl, sulfonyl and sulfonamido;
R²⁸, R²⁹, R³⁰, R³², R³³, R³⁴, R³⁶ and R³⁷ are each optionally present and when no double bond is present to the carbon atom to which it is bonded in the ring, two groups are optionally present, and, when present, each is substituted for one hydrogen present in the ring, or when no double bond is present to the carbon atom to which it is bonded in the ring, is substituted for one or both of the two hydrogen atoms present on the ring and each is independently selected from the group consisting of alkyl, substituted alkyl, cycloalkyl, substituted cycloalkyl, a heterocyclic group, a substituted heterocyclic group, aryl, substituted aryl, heteroaryl, substituted heteroaryl, hydroxy, alkoxy, aryloxy, oxo, amino, formyl, acyl, carboxy, carboxyalkyl, carboxyaryl, amido, carbamoyl, guanidino, ureido, amidino, mercapto, sulfinyl, sulfonyl, sulfonamide and, only if a double bond is present, halogen; and
R⁴⁰ and R⁴¹ are each independently hydrogen, C₁-C₁₀-alkyl, substituted C₁-C₁₀-alkyl, R^{AA} as defined above, or alternatively R⁴⁰ and R⁴¹ together form a 3- to 12-membered cyclic ring optionally comprising one or more heteroatoms selected from the group consisting of O, S and N wherein the ring is optionally substituted with R⁸ as defined previously, or alternatively one of R⁴⁰ and R⁴¹ is hydroxy, alkoxy, aryloxy, amino, mercapto, carbamoyl, amidino, ureido or guanidino, while the other is hydrogen, C₁-C₁₀-alkyl or substituted C₁-C₁₀-alkyl, except when the carbon to which R⁴⁰ and R⁴¹ are bonded is also bonded to another heteroatom;
with the proviso that T is not an amino acid residue, dipeptide fragment, tripeptide fragment or higher order peptide fragment comprising standard amino acids.

12. The method according to claim 11, wherein the compound is selected from following formula (Xa), (Xb), and (Xc):

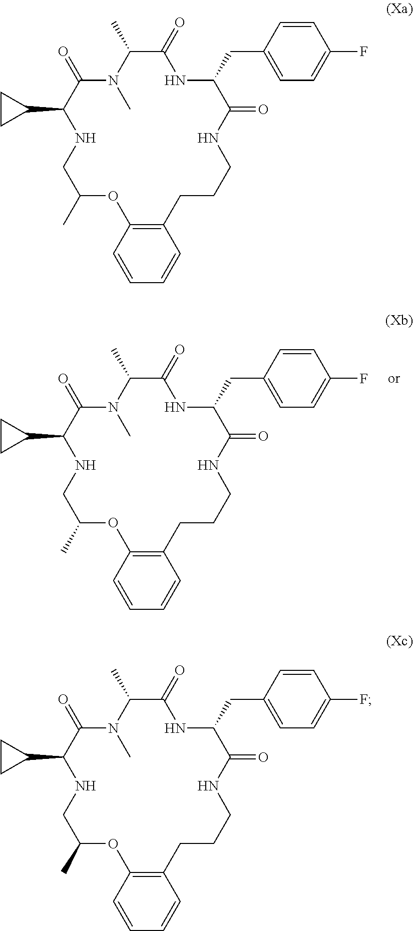 [SMILES: CC1CN[C@@H](C2CC2)C(=O)N(C)[C@H](C)C(=O)N[C@H](Cc2ccc(F)cc2)C(=O)NCCCc2ccccc2O1 ; C[C@@H]1CN[C@@H](C2CC2)C(=O)N(C)[C@H](C)C(=O)N[C@H](Cc2ccc(F)cc2)C(=O)NCCCc2ccccc2O1 ; C[C@@H]1C(=O)N[C@H](Cc2ccc(F)cc2)C(=O)NCCCc2ccccc2O[C@@H](C)CN[C@@H](C2CC2)C(=O)N1C]

13. A method for the treatment of achlorhydria, which comprises administering an effective amount of one or more selected from the group consisting of a compound of the formula (XI), a racemic-diastereomeric mixture, and an optical isomer of the said compound, and a pharmaceutically-acceptable salt and a prodrug thereof to a human or an animal:
A-B—C-D(-E)ₚ (XI)
wherein p is 0 or 1;
A is hydrogen or R¹—(CH₂)_{q}—(X)ᵣ—(CH₂)ₛ—CO—, wherein
q is 0 or an integer between 1 and 5;
r is 0 or 1;
s is 0 or an integer between 1 and 5;
R¹ is hydrogen, imidazolyl, guanidino, piperazino, morpholino, piperidino or N(R²)—R³, wherein each of R² and R³ is independently hydrogen or C₁-C₁₀-alkyl optionally substituted by one or more hydroxyl, pyridinyl or furanyl groups; and
X, when r is 1, is —NH—, —CH₂—, —CH═CH—, 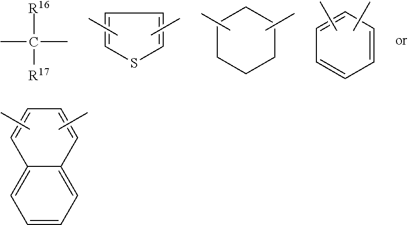
wherein each of R¹⁶ and R¹⁷ is independently hydrogen or C₁-C₁₀-alkyl;
B is (G)ₜ-(H)ᵤ wherein
t is 0 or 1;
u is 0 or 1;
G and H are amino acid residues selected from the group consisting of a natural L-amino acid or its corresponding D-isomers, and non-natural amino acids such as 1,4-diaminobutyric acid, amino-isobutyric acid, 1,3-diaminopropionic acid, 4-aminophenylalanine, 3-pyridylalanine, 1,2,3,4-tetrahydroisoquinoline-3-carboxylic acid, 1,2,3,4-tetrahydronorharman-3-carboxylic acid, N-methylanthranilic acid, anthranilic acid, N-benzylglycine, 3-amino-3-methylbenzoic acid, 3-amino-3-methyl butanoic acid, sarcosine, nipecotic acid or iso-nipecotic acid;
and wherein, when both t and u are 1, the amide bond between G and H is optionally substituted by
Y—NR¹⁸—, wherein Y is —CO— or —CH₂—, and R¹⁸ is hydrogen, C₁-C₁₀-alkyl or lower aralkyl;
C is a D-amino acid residue of formula —NH—CH((CH₂)_{w}—R⁴)—CO— wherein
w is 0, 1 or 2; and
R⁴ is selected from the group consisting of 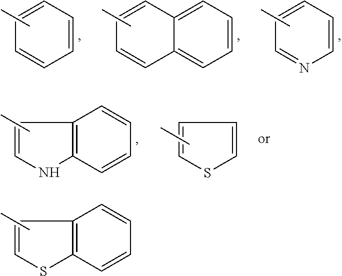
each of which is optionally substituted with halogen, C₁-C₁₀-alkyl, C₁-C₁₀-alkyloxy, C₁-C₁₀-alkylamino, amino or hydroxy;
D, when p is 1, is a D-amino acid of formula —NH—CH((CH₂)ₖ—R⁵)—CO— or, when p is 0, D is —NH—CH((CH₂)_{I}—R⁵)—CH₂—R⁶ or —NH—CH((CH₂)ₘ—R⁵)—CO—R⁶, wherein
k is 0, 1 or 2;
1 is 0, 1 or 2;
m is 0, 1 or 2;
R⁵ is selected from the group consisting of 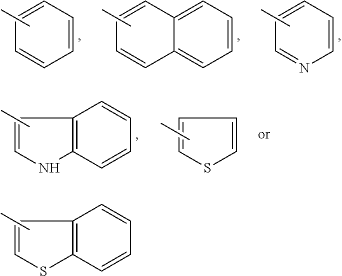
each of which is optionally substituted with halogen, alkyl, alkyloxy amino or hydroxy; and
R⁶ is piperazino, morpholino, piperidino, —OH or —N(R⁷)—R⁸, wherein each of R⁷ and R⁸ is independently hydrogen or C₁-C₁₀-alkyl;
E, when p is 1, is —NH—CH(R¹⁰)—(CH₂)ᵥ—R⁹, wherein v is 0 or an integer between 1 and 8;
R⁹ is hydrogen, imidazolyl, guanidino, piperazino, morpholino, piperidino, 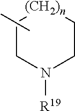
wherein n is 0, 1 or 2, and R¹⁹ is hydrogen or C₁-C₁₀-alkyl, 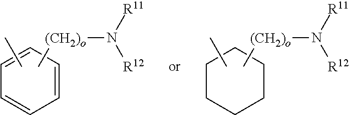
wherein o is an integer from 1 to 3,
or N(R¹¹)—R¹², wherein each of R¹¹ and R¹² is independently hydrogen or C₁-C₁₀-alkyl, or 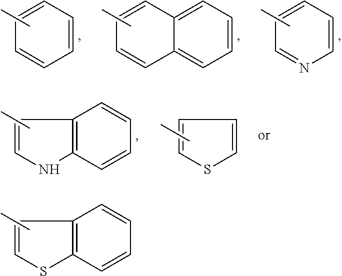
each of which is optionally substituted with halogen, alkyl, alkyloxy, amino, alkylamino, hydroxy, or the Amadori rearrangement product from an amino group and a residue formed by eliminating hydrogen from a hexapyranose or a hexapyranosyl-hexapyranose
and
R¹⁰, when p is 1, is selected from the group consisting of —H, —COOH, —CH₂—R¹³, —CO—R¹³ or —CH₂—OH, wherein
R¹³ is piperazino, morpholino, piperidino, —OH or —N(R¹⁴)—R¹⁵, wherein each of R¹⁴ and R¹⁵ is independently hydrogen or C₁-C₁₀-alkyl;
the amide bond between B and C or, when t and u are both 0, between A and C being optionally substituted by R¹⁸ or
Y—NR¹⁸—, wherein Y is —CO— or —CH₂—, and R¹⁸ is hydrogen, C₁-C₁₀-alkyl or lower aralkyl, or, when p is 1, the amide bond between D and E being optionally substituted by Y—NR¹⁸—, wherein Y and R¹⁸ are as indicated above;
or a pharmaceutically acceptable salt thereof.

14. The method according to claim 13, wherein the compound is following formula (XII):

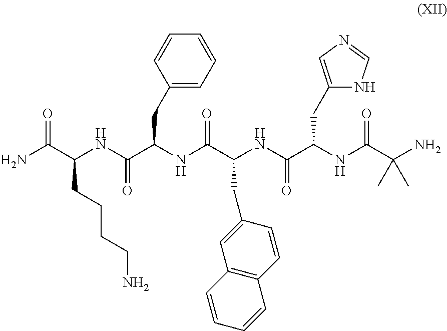 [SMILES: CC(C)(N)C(=O)N[C@@H](Cc1cnc[nH]1)C(=O)N[C@H](Cc1ccc2ccccc2c1)C(=O)N[C@H](Cc1ccccc1)C(=O)N[C@@H](CCCCN)C(N)=O]

15. The method according to any one of claims 1, 2, 5, 7, 9, 11 and 13, wherein the molecular weight of the compound is lower than 800.

16. The method according to any one of claims 1, 2, 5, 7, 9, 11 and 13, wherein the achlorhydria is age-associated achlorhydria that accompanies the aging process; chronic gastritis-associated achlorhydria; anemic achlorhydria that accompanies the anemic condition; partial gastrectomy-associated achlorhydria; calcium absorption-associated achlorhydria; vitamin D absorption-associated achlorhydria; calcitonin synthesis-associated achlorhydria; and drug-induced achlorhydria.

17. A method for the treatment of achlorhydria, which comprises administering and effective amount of a compound or a pharmaceutically acceptable salt thereof identified in any one of claims 1, 2, 5, 7, 9, 11 and 13 in combination with one or more second active agents.

18. The method according to claim 17, wherein the second active agents are any one of agents selected from:
(i) a histamine H₂ receptor antagonists, (ii) a proton pump inhibitors, (iii) an oral antacid mixture, (iv) a mucosal protective agent, (v) an anti-gastric agent, (vi) a 5-HT3 antagonist, (vii) a 5-HT4 agonist, (viii) laxative, (ix) a GABAB agonist, (x) a GABAB antagonist, (xi) a calcium channel blocker, (xii) a dopamine antagonist, (xiii) a Tachykinin (NK) antagonist, (xiv) a Helicobacter pylori infection agent, (xv) a nitric oxide synthase inhibitor, (xvi) a vanilloid receptor 1 antagonist, (xvii) a muscarinic receptor antagonist, (xviii) a calmodulin antagonist, (xix) a potassium channel agonist, (xx) a beta-1 agonist, (xxi) a beta-2 agonist, (xxii) a beta agonist, (xxiii) an alpha 2 agonist, (xxiv) an endothelin A antagonist, (xxv) an opioid μ agonist, (xxvi) an opioid μ antagonist, (xxvii) a motilin agonist, (xxviii) a ghrelin agonist, (xxix) an AchE release stimulant, (xxx) a CCK-B antagonist, (xxxi) a glucagon antagonist, (xxxii) piperacillin, Ienampicillin, tetracycline, metronidazole, bithmuth citrate and bithmuth subsalicylate, (xxxiii) a Glucagon-like peptide-1]
(GLP-1) antagonist, (xxxiv) a small conductance calcium-activated potassium channel 3 (SK-3) antagonist, (xxxv) a mGluR5 antagonist, (xxxvi) a 5-HT3 agonist, (xxxvii) a mGluR8 agonist, (xxxviii) a chemotherapeutic agent, (xxxix) an immunotherapeutic agent, (xL) a drug for cachexia, (xLi) a diuretic agent, and (xLii) an antidepressant.

19. (canceled)

20. A pharmaceutical composition comprising a compound or a pharmaceutically acceptable salt thereof identified in any one of claims 1, 2, 5, 7, 9, 11 and 13 for the treatment of achlorhydria.

21. A kit for the treatment of achlorhydria, comprising a compound or a pharmaceutically acceptable salt thereof identified in any one of claims 1, 2, 5, 7, 9, 11 and 13.

22. A kit for the treatment of achlorhydria, comprising a compound or a pharmaceutically acceptable salt thereof identified in an one of claims 1, 2, 5, 7, 9, 11 and 13, at least one second active agent, and a container.

23. A commercial package comprising a pharmaceutical composition containing a compound or a pharmaceutically acceptable salt thereof identified in any one of claims 1, 2, 5, 7, 9, 11 and 13 and a written matter associated with said pharmaceutical composition, the written matter stating that said pharmaceutical composition can or should be used for treating achlorhydria.
